(19) United States
(12) Patent Application Publication
(10) Pub. No.: US 2023/0049682 A1
(43) Pub. Date: Feb. 16, 2023

(54) Protein Separation

(71) Applicant: Technological University Dublin, Dublin (IE)

(72) Inventors: Andrew Knox, Dublin (IE); Jamie Song, Dublin (IE)

(21) Appl. No.: 17/758,181

(22) PCT Filed: Dec. 31, 2020
(86) PCT No.: PCT/EP2020/088089

§ 371 Date: Jun. 29, 2022

(30) Foreign Application Priority Data
Dec. 31, 2019 (GB) 1919461.2

(51) Int. Cl. C07K 1/34; C07K 14/77; C07K 14/79; C07K 14/81; C12N 9/36; A23J 1/09

(52) U.S. Cl. CPC C07K 1/34; C07K 14/77; C07K 14/79; C07K 14/8117; C12N 9/2462; A23J 1/09; C12Y 302/01017

(57) ABSTRACT

The invention provides a method of extracting protein from a protein source material such as egg white material. The method comprises contacting a crosslinked alginate-based carrier with the protein source material and allowing the protein to bind to the carrier, so as to form a protein-loaded carrier product. The method then comprises separating the protein-loaded carrier product from the remaining protein source material.

### FIELD OF THE INVENTION

The present invention relates to a method of extracting a protein from a protein source material, using an alginate-based carrier to form a protein-loaded carrier. In particular, in one embodiment the present invention relates to a method of extracting a protein from egg white material, using an alginate-based carrier to form a protein-loaded carrier. The present invention also relates to an alginate-based carrier and a method of making the alginate-based carrier.

### BACKGROUND OF THE INVENTION

Naturally available proteins have a range of useful applications. For example, they have been found to have utility in the biomedical, pharmaceutical, agricultural and food industries.

Animal proteins are widely used for the formation of protein particles in the food industry, e.g., casein, whey protein, gelatin, fibroin, and egg proteins.

Proteins isolated from milk are one of the most commonly used proteins from animal sources, e.g., caseins and whey proteins. Whey proteins are widely used in the food industry due to their high nutritional value and their characteristics in terms of emulsification, gelling, foaming, and thickening. Albumins are used for the production of microparticles or nanoparticles, as they are biodegradable and nontoxic. Gelatin is also a useful material to produce food-grade protein particles. Fibroin has been used in biomedical applications because of its biocompatibility, biodegradability, antimicrobial properties, and high thermal stability.

Ovalbumin is the major protein found in egg white, making up about 55wt % of the total protein found in egg white. It has anticancer, antihypertensive, antimicrobial, antioxidant, and immune-modulating properties.

Lysozyme is another protein found in egg white, and has been used to grow functional materials for catalysis and biomedical applications. It also exhibits antibacterial properties, which has led to its use as a preservative in the food industry.

The protein ovotransferrin (conalbumin) makes up about 12-13wt % of the total protein found in egg white and is highly commercially valuable. For example, it has antimicrobial, iron-transporting, anticancer, antioxidative, antihypertensive, and immunomodulatory properties.

As such, proteins from egg white find a wide variety of uses. Such proteins may be attractive for use due to their natural and sustainable source. In addition, sourcing protein from egg white allows “waste” eggs or egg whites to be put to good use, rather simply being disposed of.

There is therefore a need for an efficient purification system for egg white proteins, which can play a major role in the biomedical, pharmaceutical, agricultural and food industries.

Common techniques to purify egg white proteins include size-exclusion chromatography and ion-exchange chromatography. Omana et al, J. Chromatogr. B 2010, 878 (21), 1771-1776 describes the co-extraction of egg white proteins using ion-exchange chromatography from ovomucin-removed egg whites.

However, chromatographic methods require multiple expensive steps to obtain a purified protein and may include the use of potentially toxic materials.

It has been reported that conventional processes for protein extraction account for approximately 50-80% of the overall production cost of such proteins. Therefore, a cost effective method of extracting protein from egg white remains desirable.

An environmentally friendly and low toxicity method of extracting protein from egg white likewise remains desirable.

Similar issues apply to extracting other useful natural proteins in a selective, environmentally friendly and low toxicity manner.

For example, aprotinin (also known as bovine pancreatic trypsin inhibitor or BPTI) is a useful natural protein. It inhibits trypsin and related proteolytic enzymes. Aprotinin slows down fibrinolysis, the process that leads to the breakdown of blood clots. It is used as a medication to reduce bleeding, e.g. during cardiopulmonary bypass, and can decrease the need for blood transfusions during surgery, as well as reducing end-organ damage due to hypotension as a result of marked blood loss. Aprotinin is present in bovine lung tissue and can be extracted from bovine lung solution.

Lysozyme is also found in milk, and it would be useful to be able to extract lysozyme selectively from milk. In particular, work has been done on increasing the levels of lysozyme in milk from farm animals, such as cows and goats, by genetic modification. Thus there is a need to selectively extract lysozyme from milk obtained from such transgenic farm animals.

The present invention has been devised with the foregoing in mind.

### SUMMARY OF THE INVENTION

According to a first aspect, the present invention provides a method of extracting protein from protein source material, wherein the method comprises: a) contacting a crosslinked alginate-based carrier with the protein source material and allowing the protein to bind to the carrier, so as to form a protein-loaded carrier product; and b) separating the protein-loaded carrier product from the remaining protein source material. The protein source material comprises a mixture of two or more (such as three or more, or four or more) naturally-occurring proteins and water. It may optionally further comprise one or more of minerals, fats, vitamins and glucose.

The protein source material is of natural origin. The protein source material is suitably of animal origin, for example it may be an animal egg product, e.g. an egg white material, or may be an animal milk product, or may be an animal organ extract, e.g. an animal lung extract.

In one preferred embodiment of the first aspect, the present invention provides a method of extracting protein from egg white material, wherein the method comprises: a) contacting a crosslinked alginate-based carrier with the egg white material and allowing the protein to bind to the carrier, so as to form a protein-loaded carrier product; and b) separating the protein-loaded carrier product from the remaining egg white material.

According to a second aspect, the present invention provides a protein-loaded carrier product, comprising protein bound to a crosslinked alginate-based carrier, wherein the protein-loaded carrier product is obtainable by (e.g. has been produced by) the method of the first aspect.

The present inventors have determined that a crosslinked alginate-based carrier can be used to successfully extract protein from egg white material. The protein as extracted may be in pure form or substantially pure form. It may be that the method of the first aspect is repeated on the remaining egg white material, to extract a different protein. The method could be repeated multiple times to obtain two or more, e.g. three or more, different proteins. In one embodiment, both ovalbumin and lysozyme are extracted.

A surprising and beneficial finding of the present invention is that by controlling the nature of the alginate-based carrier used and/or the conditions under which the carrier is exposed to the protein source material, e.g. egg white material, proteins can be extracted selectively. In this regard, selective extraction may refer to a specific protein being preferentially or solely extracted; in particular it may be that the specific protein makes up 75wt % or more, such as 80wt % or more, or 85wt % or more, or 90wt % or more, or 95wt % or more, or 98wt % or more, or 99wt % or more, of the extracted protein.

It is desirable to selectively extract a single type of protein from a complex mixture of proteins, such as egg white. However, this presents significant technical challenge as compared to the absorption of an isolated protein onto any given carrier.

Even where it has been shown that a carrier can absorb a particular protein, it cannot be assumed that the protein will selectively be absorbed over any other proteins present in a complex mixture, such as egg white.

For example, X Li et al., Biochem. Eng. J., 126 (2017) 50-57 describes the absorption of already-purified protein solutions, such as lysozyme, onto beads from a buffered solution. However, there is no teaching about selective extraction of lysozyme from a complex mixture such as egg white. Nor is there any teaching about the separation of lysozyme from any other egg white proteins.

In the present invention, in one preferred embodiment the alginate-based carrier is covalently crosslinked and the functional groups on the alginate-based carrier are used to control the protein that is extracted selectively.

In particular, it has been found that by using a covalently crosslinked alginate-based carrier with carboxylate functional groups, lysozyme may be selectively extracted. Meanwhile, by using a covalently crosslinked alginate-based carrier with amine functional groups, ovalbumin may be selectively extracted.

Unexpectedly, it has been determined that covalently crosslinked alginate-based carrier with amine functional groups can be used to extract relatively large amounts of ovalbumin from egg white. Such beads can have a high capacity for binding ovalbumin, e.g. a binding capacity of 14.5 mg/ml has been achieved. These beads are also mechanically very stable. This is advantageous in that it allows for the carrier to be easily separated from the residual egg white material.

Therefore, in one preferred embodiment of the method of the first aspect, the protein is ovalbumin, and the crosslinked alginate-based carrier is an amine-functionalised carrier comprising a crosslinked alginate having amine functional groups thereon.

It has been found that covalently crosslinked alginate-based carrier with carboxylate functional groups can be used to extract relatively large amounts of lysozyme from egg white. Such beads can have a good capacity for binding lysozyme, e.g. a binding capacity of 1.37 mg/ml has been achieved. These beads are also mechanically very stable. This is advantageous in that it allows for the carrier to be easily separated from the residual egg white. Unexpectedly, particularly good results can be achieved by lowering the pH of the egg white material, e.g. to be in the pH range of 4-6, especially 5.

In contrast, Brassesco, et al, Int. J. Biol. Macromol. 2017, 96, 111-117, uses epichlorohydrin cross-linked alginate-guar gum beads at a pH of 7 to adsorb lysozyme, using phosphate buffer pH 7.0 and HCl to achieve this pH.

In the present invention it has also, unexpectedly, been found that a carrier which is ionically crosslinked calcium alginate formed using an alginate concentration from 1% to 6% (w/v) and a calcium concentration from 6% to 12% (w/v) is selective for ovalbumin, especially when the pH of the egg white material is adjusted to be high, e.g. 8-10. The calcium alginate may be in the form of beads and these may be obtainable by (e.g. may have been prepared by) dropping a solution of alginate into a solution of calcium ions.

It has also, unexpectedly, been found that a carrier which is ionically crosslinked calcium alginate formed using an alginate concentration from 4 to 8% (w/v) and a calcium concentration from 1 to 5% (w/v) is selective for lysozyme, especially when the pH of the egg white material is adjusted to be low, e.g. 4-6. The calcium alginate may be in the form of beads and these may be obtainable by (e.g. may have been prepared by) dropping a solution of alginate into a solution of calcium ions.

Other known techniques for making crosslinked calcium alginate beads may also be contemplated, such as spray drying, for example as described in Brassesco et al, Process Biochem. 2019, 80, 157-163.

In the present invention, the protein is extracted from the protein source material, e.g. egg white material, in the form of the protein-loaded carrier product, i.e. there is the extracted protein bound to the crosslinked alginate-based carrier. It will be appreciated that this extracted protein can subsequently be removed from the carrier to provide protein in unbound form.

Thus, according to a third aspect, the present invention provides a method of providing protein in unbound form, the method comprising: i) providing protein-loaded carrier product as defined in the second aspect; and ii) removing protein from the crosslinked alginate-based carrier so as to provide protein in unbound form. In one embodiment, step i) comprises carrying out the method of the first aspect.

According to a fourth aspect, the present invention provides protein in unbound form obtainable by (e.g. produced by) the method of the third aspect.

According to a fifth aspect, the present invention provides a method of producing a covalently crosslinked alginate carrier, the method comprising: a) providing an ionically crosslinked alginate; and b) reacting the ionically crosslinked alginate with 1M-3M epichlorohydrin, so as to produce a covalently crosslinked alginate carrier.

According to a sixth aspect, the present invention provides a covalently crosslinked alginate carrier obtainable by (e.g. produced by) the method of the fifth aspect.

According to a seventh aspect, the present invention provides a method of producing an amine-functionalised carrier, the method comprising: i) providing a crosslinked alginate; and ii) reacting the crosslinked alginate with an azide moiety so as to produce an amine-functionalised alginate carrier.

The reaction of the crosslinked alginate, specifically carboxylic acid moieties of the crosslinked alginate, with azide to produce amines is a Schmidt reaction. In a preferred embodiment the azide reacts with carboxylic acid moieties on the crosslinked alginate. Therefore, some or all of the carboxylic acid moieties on the crosslinked alginate are converted into amine moieties.

Other methodologies have previously been used to functionalise alginates by conjugation to the acidic group; for example see Liu, S. et al, Biomacromolecules 2015, 16 (9), 2556-2571. However, previous methodologies have not used a Schmidt reaction. Therefore, in such previous methodologies, carboxylic acid moieties on the crosslinked alginate were not converted into amine moieties. Conjugation to the acidic group is clearly different from conversion of the acidic group.

Zhang, N., et al., Int. J. Pharm. 2010, 393 (1-2), 212-218 describes alginate/chitosan nanoparticles and their use in loading and releasing insulin. However, amine functional groups are not provided on a crosslinked alginate in this methodology. There is no conversion of carboxylic acid moieties on a crosslinked alginate into amine moieties. In addition, the focus is in relation to insulin; there is no teaching about how to achieve selective absorption of proteins from egg white.

Converting the acidic functionality of an alginate to an amine will significantly alter its properties. Thus the functionalised alginates of the prior art are not the same as those made by the method of the seventh aspect, and will have different properties. As discussed above, beneficial properties of the covalently crosslinked alginate-based carrier with amine functional groups according to the present invention are an unexpectedly high capacity for binding ovalbumin, and excellent mechanical stability.

In one embodiment, the crosslinked alginate provided in step i) is a covalently crosslinked alginate carrier according to the sixth aspect. In one embodiment, step i) comprises carrying out the method of the fifth aspect.

According to an eighth aspect, the present invention provides an amine-functionalised carrier comprising a crosslinked alginate having amine functional groups thereon. In one embodiment of the eighth aspect, the amine-functionalised carrier is obtainable by (e.g. produced by) the method of the seventh aspect.

The amine-functionalised carrier of the eighth aspect may be used in the method of the first aspect when the protein is ovalbumin. The amine-functionalised carrier may be obtainable by (e.g. may have been produced by) the method of the seventh aspect.

According to a ninth aspect, the present invention provides the use of a crosslinked alginate based carrier to selectively extract ovalbumin from egg white material, wherein the carrier is (i) covalently crosslinked alginate having amine functional groups; or (ii) ionically crosslinked calcium alginate formed using an alginate concentration from 1% to 6% (w/v) and a calcium concentration from 6% to 12% (w/v). The calcium alginate may be in the form of beads and these may be obtainable by (e.g. may have been prepared) by dropping a solution of alginate into a solution of calcium ions.

According to a tenth aspect, the present invention provides the use of a crosslinked alginate based carrier to selectively extract lysozyme from egg white material or from milk-based material, wherein the carrier is (i) covalently crosslinked alginate having carboxylate functional groups; or (ii) ionically crosslinked calcium alginate formed using an alginate concentration from 4 to 8% (w/v) and a calcium concentration from 1 to 5% (w/v). The calcium alginate may be in the form of beads and these may be obtainable by (e.g. may have been prepared) by dropping a solution of alginate into a solution of calcium ions.

According to an eleventh aspect, the present invention provides the use of a crosslinked alginate based carrier to selectively extract aprotinin from bovine lung solution, wherein the carrier is (i) covalently crosslinked alginate having carboxylate functional groups; or (ii) ionically crosslinked calcium alginate formed using an alginate concentration from 4 to 8% (w/v) and a calcium concentration from 1 to 5% (w/v). The calcium alginate may be in the form of beads and these may be obtainable by (e.g. may have been prepared) by dropping a solution of alginate into a solution of calcium ions.

In all aspects of the invention, the crosslinked alginate-based carrier is preferably in the form of beads, in particular calcium alginate beads. This provides a high surface area on which the protein can bind.

In one embodiment of the invention, the calcium alginate beads comprise 0.25 ppm or more Ca²⁺, such as 0.5 ppm or more, or 0.75 ppm or more, or 1 ppm or more, or 2 ppm or more; e.g. from 0.25 ppm to 10 ppm, or from 0.5 to 8 ppm, or from 0.75 to 6 ppm, or from 1 to 5 ppm. This can be determined using ion-coupled plasma atomic emission spectroscopy (ICP-AES).

When the crosslinked alginate-based carrier is calcium alginate, the skilled person will appreciate that ICP-AES (OES) can be used to measure the concentrations of Ca²⁺. Quantification of alginate can be achieved via a number of known techniques, for example: preliminary enzymatic depolymerisation; or chemical hydrolysis of alginate to release simple carbohydrates as uronic acids, to then be detected by colorimetric methods or chromatographic methods using ion-exchange or size-exclusion chromatography; or HPLC quantification of total alginate, e.g. as in Awad et al, Journal of Chromatographic Science, 2013; 51:208-214.

A benefit of the present invention is the bioavailability of the alginate material used. Thus the invention provides a way to obtain useful protein materials in an environmentally friendly manner. This is, in particular, the case when the protein source material is egg white material, which is also readily available.

A further benefit of the present invention is the low toxicity of the materials used.

Another benefit of the present invention is the ability to obtain good yields of relatively pure protein, where the only impurities are other proteins.

The proteins as obtained by the invention are suitable for use in the biomedical, pharmaceutical, agricultural and food industries.

Yet another benefit of the present invention is the ability to obtain ovalbumin, which is known to be difficult to isolate.

### DETAILED DESCRIPTION OF THE INVENTION

Protein Source Material

The protein source material comprises a mixture of two or more (such as three or more) naturally-occurring proteins and water. It may further comprise one or more of minerals, fats, vitamins and glucose. In one embodiment it comprises a mixture of two or more (such as three or more) naturally-occurring proteins, together with water and one or more fats (lipids).

The protein source material is suitably of animal origin, for example it may be an animal egg product, e.g. an egg white material; or an animal milk product; or an animal organ extract, e.g. an animal lung extract such as bovine lung solution.

The protein source material may be used in a form as directly obtained from an animal, such as milk, or eggs, or lung lavage solution; alternatively it may be treated before use. Thus the protein source material used in the present invention may be material obtained from an animal where the material has undergone one or more treatment prior to being used.

Treatments that may be contemplated prior to use include one or more of: extraction (e.g. extraction of one or more protein), dilution (e.g. dilution with aqueous medium), and addition of one or more agent (e.g. addition of one or more water-soluble salt and/or addition of one or more surfactant and/or addition of one or more acid).

Egg White Material

One beneficial embodiment of the invention uses egg white material as the protein source material. The egg white material may be egg white or may be a treated form of egg white.

It will be understood that eggs comprise an outer protective shell that contains egg white and egg yolk. Egg white may also be known as albumen or glair. Egg yolk may also be known as vitellus. It will be understood that the white of an egg can be separated from the yolk, for example by hand or machine. Egg white generally comprises about 90wt % water and about 10 wt % protein. There may be trace amounts of minerals, fats, vitamins and glucose. The proteins found in egg white include ovalbumin, ovotransferrin, lysozyme, ovoglobulin, ovomucoid and ovomucin.

The present invention in particular relates to the extraction of protein from egg white material. The egg white material may derive from any suitable eggs, for example poultry eggs, such as eggs from chickens, ducks, geese, turkey, quails, guinea fowl, or the like. The source of the eggs is not limited provided that the egg white of the eggs contains one or more protein that is desired to be obtained as a useful material in extracted form.

In one embodiment the egg white material is egg white or consists essentially of egg white. For example, egg white may have been obtained by separating egg white from egg yolk as obtained out of one or more eggs, such as poultry eggs.

However, it will be appreciated that the egg white material used in the present invention may be egg white that has undergone one or more treatment prior to being used.

Treatments that may be contemplated prior to the egg white being used include one or more of: extraction (e.g. extraction of one or more protein), dilution (e.g. dilution with aqueous medium), and addition of one or more agent (e.g. addition of one or more water-soluble salt and/or addition of one or more surfactant and/or addition of one or more acid).

In one embodiment, it may be that the egg white has been treated so that its pH is in the range of from 4 to 9, such as from 5 to 9.

In one embodiment, the egg white material is egg white that has already been subjected to an extraction process. For example, the egg white material may be egg white that has had one or more proteins extracted from it.

For example, in one embodiment, the method of the first aspect may be carried out two or more times, each time using a different alginate-based carrier and/or using different conditions for exposing the carrier to the egg white material, so as to selectively extract different proteins in turn. Thus it may be that the method of the first aspect is carried out a first time, to extract a first protein, using egg white material that is egg white, and then the method is carried out a second time, to extract a second protein, using egg white material that is egg white minus the first protein. Optionally the method may be carried out a third time, to extract a third protein, using egg white material that is egg white minus the first protein and minus the second protein. In general, the method may be repeated until all proteins of interest have been extracted.

In one embodiment, the egg white material is egg white that has been diluted. Egg white may, for example, be diluted with an aqueous medium. The option of dilution applies to egg white and to egg white that has undergone an extraction process.

It will be appreciated that dilution may assist with the processing and handling ability of the egg white. There are no specific limits on the ratio, vol/vol, of egg white (which may optionally be egg white that has undergone another treatment, e.g. an extraction process) to aqueous medium, but it may, for example, be 100:1 or less, such as 50:1 or less, or 25:1 or less, or 20:1 or less, e.g. 15:1 or less, or 10:1 or less, preferably 5:1 or less, or 1:1 or less, such as 1:2 or less, or 1:3 or less, or 1:4 or less. In one embodiment the ratio, vol/vol, of egg white (which may optionally be egg white that has undergone another treatment, e.g. an extraction process) to aqueous medium is from 100:1 to 1:100, such as from 50:1 to 1:50 or from 30:1 to 1:30, preferably from 10:1 to 1:30, such as from 5:1 to 1:20 or from 1:1 to 1:15 or 1:2 to 1:10.

The aqueous medium may solely be water, or may optionally include additional components. In one embodiment, the aqueous medium contains water in an amount of 80% or more by weight, such as 85% or more, or 90% or more, or 95% or more, such as 98% or more, or 99% or more by weight.

It may be that the egg white used as the egg white material in the present invention has had one or more water-soluble salt added thereto. In one embodiment, the water-soluble salt can be added as part of a dilution process, e.g. before, during or after a dilution process. The aqueous medium used for dilution may, in one embodiment, contain one or more water-soluble salt.

Suitable water-soluble salts include those where the anions are halides (e.g. fluoride, chloride, bromide and iodide), sulfates and phosphates. It may be that the cation of the salt is selected from lithium, sodium and potassium. In one embodiment the one or more water-soluble salt is selected from sodium chloride, sodium bromide, sodium iodide, potassium chloride, potassium bromide and potassium iodide.

The aqueous medium may contain one or more water-soluble salt in a concentration of 0.01 mM or more, or 0.05 mM or more, such as 0.1 mM or more, or 0.5 mM or more, or 1 mM or more. In one embodiment the aqueous medium contains one or more water-soluble salt in a concentration of 5 mM or more, such as 10 mM or more, or 20 mM or more, such as 30 mM or more, or 40 mM or more, for example 50 mM or more or 100 mM or more, or 500 mM or more. In one embodiment the aqueous medium may contain one or more water-soluble salt in a concentration of from 0.01 mM to 5M, such as from 1 mM to 2M, or from 5 mM to 1M, such as from 10 mM to 750 mM or from 20 mM to 500 mM.

Whilst it is clearly convenient to provide the one or more water-soluble salt in the aqueous medium as provided to carry out the dilution process, it will be appreciated that one or more water-soluble salt may be added before, during or after the dilution process so as to achieve a desired concentration of water-soluble salt in the aqueous medium as used in the dilution.

The protein ovotransferrin (conalbumin) makes up about 12-13wt % of the total protein found in egg white and is highly commercially valuable. For example, it has antimicrobial, iron-transporting, anticancer, antioxidative, antihypertensive, and immunomodulatory properties.

In one embodiment, the presence or absence of a salt solution is used to control the protein that is extracted selectively. It has been found that by using an alginate-based carrier in a salt solution, ovotransferrin may be selectively extracted. The alginate-based carrier used may be ionically or covalently crosslinked. In one embodiment it has carboxylate functional groups.

As such, in the method of the first aspect, the aqueous medium may contain one or more water-soluble salt, such as sodium chloride. Optionally, the crosslinked alginate based carrier is ionically crosslinked calcium alginate having carboxylate functional groups and the protein is ovotransferrin.

In one preferred embodiment the egg white starting material includes egg white (which may optionally be egg white that has undergone another treatment, e.g. an extraction process) that has been diluted by an aqueous medium in a vol/vol ratio of from 100:1 to 1:100, e.g. from 10:1 to 1:30, such as from 5:1 to 1:20 or from 1:1 to 1:15 or 1:2 to 1:10, wherein the aqueous medium contains one or more water-soluble salt (e.g. selected from sodium chloride, sodium bromide, sodium iodide, potassium chloride, potassium bromide and potassium iodide) in a concentration of 0.01 mM or more, e.g. from 0.01 mM to 5M, such as 5 mM or more, e.g. from 5 mM to 1M, such as from 10 mM to 750 mM or from 20 mM to 500 mM. Such an embodiment is in particular useful for the selective extraction of ovotransferrin.

It may be that the egg white used as the egg white material in the present invention (which may optionally be egg white that has undergone another treatment, e.g. an extraction process) has had one or more surfactant added thereto. In one embodiment, the surfactant can be added as part of a dilution process, e.g. before, during or after a dilution process. The aqueous medium used for dilution may, in one embodiment, contain one or more surfactant. However, it may be preferred to add one or more surfactant separately from any dilution process, e.g. after any dilution process. In particular, surfactant may usefully help maintain solubility of protein in an aqueous medium used for dilution.

Suitable surfactants include non-ionic surfactants, such as polysorbate-type non-ionic surfactants, e.g. esters of polyethylene glycol sorbitan with fatty acid (such as lauric acid). An example is polyethylene glycol sorbitan monolaurate with 20 ethylene oxide units (commercially available as TWEEN® 20—Croda International PLC). In general, any surfactant which is compatible with the intended end use of the protein (e.g. it may need to be pharmaceutically acceptable, or agriculturally acceptable, or safe for consumption under food standards) may be considered for use.

The skilled person will appreciate that only a low amount of surfactant is required to achieve the desired aim of maintaining solubility of the protein in any aqueous medium used for dilution. The surfactant may be added to the egg white, optionally in diluted form, in a concentration (v/v) of 100 ppm or more, such as 500 ppm or more or 1000 ppm or more, e.g. 0.01% or more or 0.1% or more. The surfactant may, for example, be added in a concentration (v/v) of 100 ppm to 1%, such as 500 ppm to 0.5%, e.g. 1000 ppm to 0.3% or 0.01% to 0.2%.

It may be that the egg white used as the egg white material in the present invention (which may optionally be egg white that has undergone another treatment, e.g. an extraction process) has had a pH adjustment agent, e.g. acid or alkali, added thereto. In one embodiment, the pH adjustment agent, e.g. acid or alkali, can be added as part of a dilution process, e.g. before, during or after a dilution process. The aqueous medium used for dilution may, in one embodiment, contain one or more pH adjustment agent, e.g. acid or alkali. However, it may be preferred to add one or more pH adjustment agent, e.g. acid or alkali, separately from any dilution process, e.g. after any dilution process.

Examples of acid that may suitably be used as a pH adjustment agent are organic acids such as citric acid, formic acid, acetic acid and lactic acid.

In this regard, it may be preferable for the egg white starting material to have a certain pH prior to contact with the carrier so as to optimize selectivity for a desired protein. In this regard, a change in pH may change the surface charge of proteins and can lead to a different degree of attraction for that protein to a given carrier.

The pH of egg white as obtained directly from eggs may commonly be from 7.6 to 9.7. In one embodiment the egg white used as the egg white material in the present invention (which may optionally be egg white that has undergone another treatment, e.g. an extraction process) has had its pH altered before use. The pH can be measured using a pH meter.

In one embodiment, the pH of the egg white material is 9 or less, or 8 or less, or 7 or less, or 6 or less. In one embodiment the pH of the egg white starting material is from 4 to 9, for example from 4 to 8, or from 4 to 7, or from 4 to 6; or it may be from 5 to 9, e.g. from 5 to 8 or from 5 to 7 or from 5 to 6; or it may be from 6 to 9 or from 6 to 8, or from 6 to 7.

It has been determined that, when extracting ovalbumin from egg white material using an ionically crosslinked calcium alginate carrier and/or a covalently crosslinked alginate carrier having amine functional groups, it is preferable for the pH of the egg white material to be higher, such as 7 or more, for example from 7 to 11, or 8 or more, for example from 8 to 10, e.g. the pH may be about 9. This has been shown to increase the selectivity with which ovalbumin can be extracted from the egg white material.

It has been determined that, when extracting lysozyme from egg white material using an ionically crosslinked calcium alginate carrier, it is preferable for the pH of the egg white material to be lower, such as 7 or less, for example from 7 to 3, or 6 or less, for example from 6 to 4, e.g. the pH may be about 5. This has been shown to increase the selectivity with which lysozyme can be extracted from the egg white material.

Milk

In another embodiment, the invention uses milk-based material as the protein source material. The milk-based material may be milk or may be a treated form of milk.

It will be understood that milk generally comprises about 85-90wt % water and about 3-5wt % protein, as well as about 2-6% fat and 3-6% lactose. There may be trace amounts of minerals. The proteins found in milk include caseins and whey proteins.

The milk-based material may derive from any suitable animal milk, for example cow's milk or goat's milk. The source of the milk is not limited provided that the milk contains one or more protein that is desired to be obtained as a useful material in extracted form. As noted above, genetic modification can increase the levels of lysozyme in milk from farm animals, such as cows and goats, and thus the milk may be milk that has been obtained from such transgenic farm animals.

The milk may undergo any of the treatments described above in relation to egg white. In one embodiment, the pH may be adjusted to assist selectivity in a manner described above in relation to egg white.

The method of the first aspect may be carried out two or more times to extract two or more different proteins, as described above in relation to egg white.

Organ Extract

In another embodiment, the invention uses an animal organ extract material as the protein source material. The animal organ extract material may be an animal organ extract or may be a treated form of animal organ extract.

It will be understood that a source of animal protein may be obtained in the form of aqueous extracts from animal organs, such as the lungs. One useful example is bovine lung solution. Bovine lung solution includes water, protein and fats. The proteins found in bovine lung solution include aprotinin.

The animal organ extract may undergo any of the treatments described above in relation to egg white. In one embodiment, the pH may be adjusted to assist selectivity in a manner described above in relation to egg white.

The method of the first aspect may be carried out two or more times to extract two or more different proteins, as described above in relation to egg white.

Crosslinked Alginate-Based Carrier

The present invention makes use of a crosslinked alginate based carrier. This may be an ionically crosslinked alginate or it may be a covalently crosslinked alginate. The alginate carrier may suitably be in the form of beads; this provides a high surface area on which the protein can bind. It may be the carrier is calcium alginate in the form of beads and these may be obtainable by (e.g. may have been prepared by) dropping a solution of alginate into a solution of calcium ions.

Alginate is a polymer of natural origin and is well known in the art. It has the advantage of being environmentally friendly as well as low in cost, and has the capacity to hold water and form gels and stable emulsions. Alginate is a water-soluble linear polysaccharide composed of 1,4-linked β-D-mannuronic and α-L-glucuronic acid residues. The uronic acids, mannuronic acid and guluronic acid, are the building blocks of alginate and these are known to have a number of free hydroxyl groups and carboxyl groups distributed along the backbone.

The gelation of alginate can be carried out under an extremely mild environment using non-toxic reactants. In particular, it is known to prepare alginate beads by extruding a solution of sodium alginate as droplets into a divalent cation solution such as Ca²⁺ or Ba²⁺.

It is known in the art to use alginate in the form of a matrix with one or more additional polysaccharide, such as guar gum or chitosan. This can be contemplated, especially if alginate forms the majority (i.e. more than 50wt %, e.g. more than 75wt %, or more than 90wt %, or more than 95wt %) of the polysaccharide matrix.

The skilled person will appreciate that alginate is anionic and in one embodiment, one or more additional anionic polysaccharides are present, e.g. selected from: carrageenan, xylan, xanthan, heparin, hyaluronic acid, and chondroitin sulfate. In one embodiment, one or more cationic polysaccharides are present, e.g. selected from: chitin, chitosan, guar gum, and cationic starches.

In one embodiment, the crosslinked alginate based carrier only comprises polysaccharides that are anionic.

In one preferred embodiment, the crosslinked alginate based carrier comprises alginate as the sole polysaccharide.

In one embodiment the crosslinked alginate based carrier is ionically crosslinked calcium alginate. In another embodiment the crosslinked alginate based carrier is ionically crosslinked barium alginate.

Ionically crosslinked calcium alginate can be prepared by adding alginate in the form of an aqueous solution (e.g. a 2-6% w/v solution) to calcium ions in the form of an aqueous solution (e.g. a 2-10% w/v solution). The alginate solution may suitably be added using a syringe with a needle.

In particular, ionically crosslinked calcium alginate can be prepared by adding sodium alginate in the form of an aqueous solution (e.g. a 2-6% w/v solution) to CaCl₂ in the form of an aqueous solution (e.g. a 2-10% w/v solution). The sodium alginate solution may suitably be added using a syringe with a needle. The CaCl₂ solution may suitably be stirred as the sodium alginate solution is added. The resulting beads can be left in the CaCl₂ solution to allow the beads to harden. For example, the beads may be left at about room temperature and for a period of time from 1 to 72 hours, e.g. 6 to 48 hours or 12 to 36 hours.

The present invention has determined that when the carrier is calcium alginate, the concentrations of alginate and calcium used to make the carrier can be controlled to further improve the selectivity of the alginate carrier for the desired protein.

If lysozyme is the desired protein, a higher alginate concentration should be used, e.g. from 4 to 8% or from 5% to 7% (w/v), and/or a lower calcium concentration should be used, e.g. from 1 to 5% or from 2% to 4% (w/v). It is also preferred that the pH of the protein source material, e.g. egg white material, is lower, e.g. from 5-6, to assist selectivity for lysozyme.

If ovalbumin is the desired protein, a lower alginate concentration should be used, e.g. from 1% to 6% or from 2 to 5% (w/v), and/or a higher calcium concentration should be used, e.g. from 6% to 12% or from 8 to 10% (w/v). It is also preferred that the pH of the protein source material, e.g. egg white material is higher, e.g. from 8-9, to assist selectivity for ovalbumin.

Thus when considering the extraction of lysozyme from egg white material using an ionically crosslinked calcium alginate carrier, it is preferable for the concentration of the alginate (e.g. sodium alginate) to be higher, such as from 4 to 8% w/v, or from 5 to 7% w/v. In the extraction of lysozyme, it is preferable for the concentration of the calcium (e.g. calcium chloride) to be lower, such as from 1 to 7% w/v, or from 1 to 5% w/v, for example from 2-4% w/v. These factors have been shown to increase the selectivity with which lysozyme can be extracted from egg white material both independently and jointly.

Meanwhile, when considering the extraction of ovalbumin from egg white material using an ionically crosslinked calcium alginate carrier, it is preferable for the concentration of the alginate (e.g. sodium alginate) to be lower, such as from 1 to 6% w/v, or from 1 to 4% w/v, or from 1 to 3% w/v, for example from 2 to 5% w/v, or from 1.5% to 2.5% w/v. In the extraction of ovalbumin, it is preferable for the concentration of the calcium (e.g. calcium chloride) to be higher, such as from 6 to 12% w/v, or from 6 to 10% w/v, or from 8 to 10% w/v. These factors have been shown to increase the selectivity with which ovalbumin can be extracted from the egg white material both independently and jointly.

Also, when considering the extraction of lysozyme from milk using an ionically crosslinked calcium alginate carrier, it is preferable for the concentration of the alginate (e.g. sodium alginate) to be higher, such as from 4 to 8% w/v, or from 5 to 7% w/v. In the extraction of lysozyme, it is preferable for the concentration of the calcium (e.g. calcium chloride) to be lower, such as from 1 to 7% w/v, or from 1 to 5% w/v, for example from 2-4% w/v.

In addition, when considering the extraction of aprotinin from bovine lung solution using an ionically crosslinked calcium alginate carrier, it is preferable for the concentration of the alginate (e.g. sodium alginate) to be higher, such as from 4 to 8% w/v, or from 5 to 7% w/v. In the extraction of lysozyme, it is preferable for the concentration of the calcium (e.g. calcium chloride) to be lower, such as from 1 to 7% w/v, or from 1 to 5% w/v, for example from 2-4% w/v.

In one embodiment, the ionically crosslinked calcium alginate is in the form of beads that have been made using alginate concentrations of 1-8% w/v and CaCl₂ concentrations of 1-12% w/v. Preferably, the ionically crosslinked calcium alginate is in the form of beads that have been made using alginate concentrations of 2-6% w/v and CaCl₂ concentrations of 2-10% w/v.

In one embodiment, the ionically crosslinked calcium alginate is in the form of beads that have been made using alginate concentrations of 4-6% w/v (e.g. 5-6) and CaCl₂ concentrations of 2-4% w/v (e.g. 2-3). In one such embodiment, the ionically crosslinked calcium alginate is in the form of beads that have been made using an alginate concentration of 6% w/v and CaCl₂ concentration of 2% w/v. These beads may in particular be useful for extracting lysozyme. They may also be useful for extracting aprotinin.

In one embodiment, the ionically crosslinked calcium alginate is in the form of beads that have been made using alginate concentrations of 2-5% w/v (e.g. 3-5) and CaCl₂ concentrations of 6-10% w/v (e.g. 8-10). In one such embodiment, the ionically crosslinked calcium alginate is in the form of beads that have been made using an alginate concentration of 5% w/v and CaCl₂ concentration of 10% w/v. These beads may in particular be useful for extracting ovalbumin.

In one embodiment the crosslinked alginate based carrier is covalently crosslinked calcium alginate. In another embodiment the crosslinked alginate based carrier is covalently crosslinked barium alginate.

Covalently crosslinked calcium alginate can be prepared from ionically crosslinked calcium alginate (e.g. prepared as above). The preferred amounts of alginate and of calcium as set out above for the ionically crosslinked calcium alginate apply equally to the covalently crosslinked calcium alginate. Thus the concentrations of alginate and calcium used to make the carrier can be controlled in the same manner as discussed above to improve the selectivity of the alginate carrier for the desired protein.

Covalently crosslinked calcium alginate can be prepared by mixing ionically crosslinked calcium alginate (e.g. prepared as above) with a bifunctional linker such as epichlorohydrin. The epichlorohydrin may suitably be provided in a solvent, such as aqueous ethanol. The epichlorohydrin may suitably be provided at a concentration of from 1M to 3M, e.g. 1M to 2M. Aqueous alkali may also be present, e.g. NaOH. The crosslinking reaction may be left to occur at about room temperature and for a period of time from 30 minutes to 48 hours, e.g. 1 to 36 hours, or 3 to 24 hours.

Preferably, the covalently crosslinked calcium alginate is in the form of beads that have been made using epichlorohydrin as the bifunctional linker, in a concentration of from 1M to 3M, preferably from 1 to 2M, e.g. from 1 to 1.5M.

It has been shown that the most total protein can be extracted using covalently crosslinked calcium alginate prepared with a 1M solution of epichlorohydrin. In addition, the most Lyz can be extracted using this carrier.

Preferably, the crosslinking reaction may be left to occur at about room temperature and for a period of time from 12 to 48 hours, e.g. 24 to 48 hours or 24 to 36 hours.

It has been shown that the amount of protein extracted increases slightly when using covalently crosslinked calcium alginate that has been crosslinked for 24 hours as compared to shorter time periods.

The chemical structure A shown below represents calcium alginate, where the alginate is ionically crosslinked. The chemical structure B shown below represents a possible structure obtainable by covalently crosslinking calcium alginate, using the bifunctional linker epichlorohydrin. Such covalent crosslinking can provide greater structural stability.

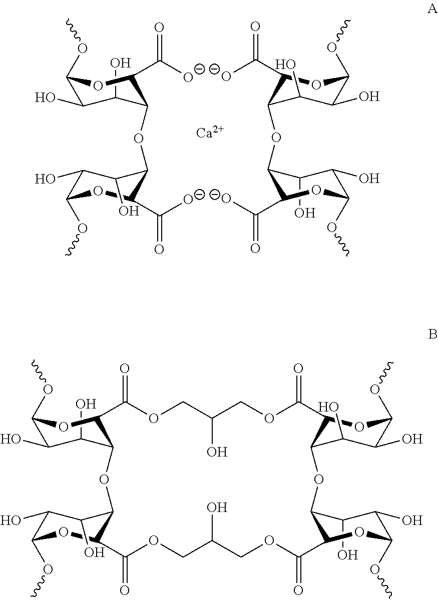

As can be seen from the above chemical structures, the ionically crosslinked alginate and the covalently crosslinked alginate have carboxylate functional groups.

In one embodiment, however, the crosslinked alginate based carrier is modified to have amine functional groups. In particular, the carboxylate group on the alginate can be converted to a primary amine. This may be achieved by using the Schmidt reaction.

Covalently crosslinked calcium alginate with amine functional groups can be prepared by mixing covalently crosslinked calcium alginate with carboxylate functional groups (e.g. prepared as above) with a solution of sodium azide. The sodium azide may suitably be provided in an amount of 0.1 to 3 g per 1 g of calcium alginate, e.g. from 0.2 to 2 g, and especially from 0.3 to 1.5 g, per 1 g of calcium alginate.

In one preferred embodiment, the sodium azide is present in an amount of from 4 to 15 mmol per 1 g of calcium alginate, such as from 5 to 13 mmol, or from 7 to 12 mmol, for example from 8.5 to 10 mmol per 1 g of calcium alginate. Such quantities have been found to increase the amount of ovalbumin extracted from egg white material.

The sodium azide may suitably be provided in acidic solution, e.g. HCl and/or H₂SO₄. It will be understood that the combination of acid and azide ions may form hydrazoic acid in situ.

The Schmidt reaction may be left to occur at about room temperature and for a period of time from 30 minutes to 12 hours or more, e.g. 1 to 8 hours or 1 to 4 hours. It may be that the reaction is left to occur at about room temperature and for a period of time from 12 hours to 24 hours or more.

In one preferred embodiment, the Schmidt reaction is left to occur at about room temperature and for a period of time of 12 hours or longer or 18 hours or longer, such as 24 hours or longer; for example a period of time from 18 hours to 72 hours, or from 24 hours to 48 hours. Periods of time that are 12 hours or longer and especially about 24 hours or longer have been found to result in a crosslinked alginate based carrier that can extract increased amounts of ovalbumin from egg white material.

It can therefore be beneficial to ensure that the reaction between the azide and the covalently crosslinked calcium alginate with carboxylate functional groups reaches completion, in order to maximize the amount of protein that can be extracted.

In one embodiment, the carrier is covalently crosslinked alginate having carboxylate functional groups and this may be used to selectively extract lysozyme. In another embodiment, the carrier is covalently crosslinked alginate having amine functional groups and this may be used to selectively extract ovalbumin. In yet another embodiment, the carrier is ionically crosslinked alginate having carboxylate functional groups and this may be used to selectively extract ovotransferrin.

In another embodiment, the carrier is covalently crosslinked alginate having carboxylate functional groups and this may be used to selectively extract aprotinin.

Selectivity for Proteins

Lysozyme has a molecular weight (Mw) of 14.4 kDa and isoelectric point (pI) of 10.7.

Ovalbumin has a higher molecular weight and lower pI (Mw of 45 kDa and pI of 4.5).

Ovotransferrin has a yet higher molecular weight (Mw of 76 kDa) and pI of 6.0.

As shown in the examples, aprotinin can be selectively extracted from bovine lung solution using similar conditions to those which selectively extract lysozyme. Aprotinin is similar to lysozyme in terms of Mw and pI, with a molecular weight of 10.9 kDa and pI of 10.5.

Thus the skilled person can identify and select further naturally occurring proteins, based on molecular weight and pI, which could be selectively extracted in a similar manner to either lysozyme or ovalbumin or ovotransferrin.

The skilled person can readily identify a protein source material containing the selected protein based on available literature.

Allowing the Protein to Bind to the Carrier (Incubation Time)

In the method of the first aspect, step a) involves contacting a crosslinked alginate-based carrier with the protein source material, e.g. egg white material, and allowing the protein to bind to the carrier, so as to form a protein-loaded carrier product. This step of allowing the protein to bind to the carrier, so as to form a protein-loaded carrier product may be referred to as the incubation step or incubation time.

In one embodiment the crosslinked alginate-based carrier and the protein source material, e.g. egg white material, are in contact for a period of time of 10 minutes or more, or 20 minutes or more, or 30 minutes or more, such as 1 hour or more, or 2 hours or more, or 3 hours or more, or 4 hours or more. In one embodiment the crosslinked alginate-based carrier and the protein source material, e.g. egg white material, are in contact for a period of time of 10 minutes to 72 hours, such as from 1 hour to 60 hours, or 2 hours to 48 hours, or 3 hours to 36 hours, such as 4 hours to 24 hours.

In one embodiment, the period of time is 30 minutes or more, or 40 minutes or more, such as 1 hour or more, or two hours or more. Such periods of time may significantly increase the amount of protein that can be extracted; this has been shown especially for the extraction of lysozyme using covalently crosslinked calcium alginate.

In one embodiment, the crosslinked alginate based carrier is ionically crosslinked calcium alginate and the protein being extracted is lysozyme and the incubation time is 12 hours or more, preferably 24 hours or more. For extracting Lyz using ionically crosslinked calcium alginate, it has been shown that there is a benefit to having a longer incubation time in order to obtain more Lyz and that this is without detriment to the purity.

In one embodiment, the crosslinked alginate based carrier is ionically crosslinked calcium alginate and the protein being extracted is ovalbumin and the incubation time is 10 minutes to 1 hour, preferably 10 minutes to 30 minutes. For extracting Oval using ionically crosslinked calcium alginate, it has been shown that there is a benefit to having a relatively short incubation time in order to obtain the best purity for the extracted Oval and this is without too much detriment to the amount extracted.

In one embodiment, the crosslinked alginate based carrier is covalently crosslinked calcium alginate (especially covalently crosslinked alginate having carboxylate functional groups) and the protein being extracted is lysozyme and the incubation time is 1 hour or more, preferably 1-2 hours or more. For extracting Lyz using covalently crosslinked calcium alginate, it has been shown that there is a benefit to using a long enough incubation time, but about 1-2 hours is both sufficient and optimal to extract Lyz.

In one embodiment, the crosslinked alginate based carrier is covalently crosslinked calcium alginate (especially covalently crosslinked alginate having amine functional groups) and the protein being extracted is ovalbumin and the incubation time is 8 hours or more, preferably 10-12 hours or more, e.g. 12-18 hours. For extracting Oval using covalently crosslinked calcium alginate, it has been shown that there is a benefit to using a long enough incubation time, but about 12 hours is both sufficient and optimal to extract Oval.

In one preferred embodiment the step of allowing the protein to bind to the carrier, so as to form a protein-loaded carrier product, involves agitation of the crosslinked alginate-based carrier and the protein source material, e.g. egg white material. For example, the crosslinked alginate-based carrier and the protein source material, e.g. egg white material, may be brought into contact in a container, such as a column, and may then be agitated by shaking the container.

In one embodiment the crosslinked alginate-based carrier and the protein source material, e.g. egg white material, are agitated for a period of time of 10 minutes or more, or 30 minutes or more, such as 1 hour or more, or 2 hours or more, or 3 hours or more, or 4 hours or more. In one embodiment the crosslinked alginate-based carrier and the protein source material, e.g. egg white material, are agitated for a period of time of 10 minutes to 72 hours, such as from 1 hour to 60 hours, or 2 hours to 48 hours, or 3 hours to 36 hours, such as 4 hours to 24 hours.

In one embodiment the step of allowing the protein to bind to the carrier, so as to form a protein-loaded carrier product, is carried out at a temperature of from 1 to 50° C., such as from 2 to 40° C., or from 3 to 30° C.; preferably the temperature is from 4 to 25° C., e.g. from 5 to 20° C., such as about 10 to 20° C.

In one embodiment, the protein-loaded carrier product is covalently crosslinked alginate having carboxylate functional groups that has lysozyme bound thereto. In another embodiment, the protein-loaded carrier product is covalently crosslinked alginate having amine functional groups that has ovalbumin bound thereto. In yet another embodiment, the protein-loaded carrier product is ionically crosslinked alginate having carboxylate functional groups that has ovotransferrin bound thereto.

In one further embodiment, the protein-loaded carrier product is covalently crosslinked alginate having carboxylate functional groups that has aprotinin bound thereto.

Separation of Protein-Loaded Carrier Product

In the method of the first aspect, step b) involves separating the protein-loaded carrier product from the remaining protein source material, e.g. egg white material.

This step may suitably be carried out by filtration. However, the skilled person will appreciate that any other suitable means of separation can be used and the invention is not limited by how the separation is achieved.

The Protein-Loaded Carrier Product

The protein-loaded carrier product may be stable for storage. For example, it may be stable at room temperature for about 5-7 days and may be stable at lower temperatures for longer. It may be that the protein-loaded carrier product is stored under refrigerated conditions, for example, for a timeframe of days or weeks or even months.

Therefore although in some embodiments the protein is removed from the protein-loaded carrier product as a next step after it is formed, it is contemplated that the protein-loaded carrier product could in itself be a useful form in which to provide and store the protein, with the protein then being removed from the protein-loaded carrier product only when it is required for use.

Removing Protein from the Crosslinked Alginate-Based Carrier

In the method of the third aspect, step ii) involves removing protein from the crosslinked alginate-based carrier so as to provide protein in unbound form.

In one embodiment, this is achieved by dissolving the protein in a solvent and then removing the resulting protein-containing solution. For example, the protein-loaded carrier product may be combined with an acidic solvent that can dissolve the protein and the resulting protein-containing solution can then be separated from the (solid) crosslinked alginate-based carrier, e.g. by filtering off the crosslinked alginate-based carrier to leave the protein-containing solution as the filtrate.

In one embodiment, the acidic solvent is aqueous NaCl, e.g. (0.1-1 M in water, such as 0.4-1M). However, other acids may also be contemplated, e.g. acetic acid.

The protein-loaded carrier product may suitably be combined with an excess of the acidic solvent to assist with the dissolving of the protein into the solvent.

In one embodiment, the protein-loaded carrier product may be combined with an excess of the acidic solvent and left for 1 minute or more, such as 2 minutes or more, or 5 minutes or more (e.g. from 1 to 30 minutes or from 2 to 15 minutes or from 5 to 10 minutes) to allow the protein to dissolve in the solvent.

In general, techniques for dissolving proteins in solvent, such as acidic solvent, are known and the skilled person will appreciate that any such techniques may be used.

It may be that the protein is subsequently freeze dried for storage. Prior to freeze drying a desalination step may be carried out to remove salt, e.g. a dialysis step may be carried out.

Crosslinking with Epichlorohydrin

In the method of the fifth aspect, an ionically crosslinked alginate is reacted with 1M-3M epichlorohydrin, so as to produce a covalently crosslinked alginate carrier.

Although epichlorohydrin is known as a crosslinking agent, in the present invention it has surprisingly been found that only the use of 1M to 3M concentration of epichlorohydrin gives covalently crosslinked alginate carriers that are both stable and able to selectively extract lysosome from egg white material. Lower concentrations give rise to carriers that are unstable, whilst higher concentrations do not effectively extract lysosome from egg white material. In one embodiment, the use of 1M concentration of epichlorohydrin is particular beneficial.

Preferably, in the method of the fifth aspect, the ionically crosslinked alginate is ionically crosslinked calcium alginate in the form of beads that have been made using alginate concentrations of 2-6% w/v and CaCl₂ concentrations of 2-10% w/v. The thus-obtained crosslinked alginate has carboxylate functional groups.

In one embodiment, which can be useful for extracting lysozyme, the ionically crosslinked calcium alginate is in the form of beads that have been made using alginate concentrations of 4-6% w/v (e.g. 5-6) and CaCl₂ concentrations of 2-4% w/v (e.g. 2-3). In one embodiment, the ionically crosslinked calcium alginate is in the form of beads that have been made using an alginate concentration of 6% w/v and CaCl₂ concentration of 2% w/v.

In one embodiment of the method of the fifth aspect, the epichlorohydrin is provided in a solvent, such as aqueous ethanol. As noted above, the epichlorohydrin is provided at a concentration of from 1M to 3M, e.g. 1M to 2M. Aqueous alkali may also be present, e.g. NaOH. The crosslinking reaction may be left to occur at about room temperature and for a period of time from 30 minutes to 48 hours, e.g. 1 to 36 hours or 3 to 24 hours.

Making an Amine-Functionalised Alginate Carrier

In the method of the seventh aspect, a crosslinked alginate is reacted with an azide moiety, e.g. an azide salt, so as to produce an amine-functionalised alginate carrier.

Producing an amine-functionalised crosslinked alginate in this manner (via a Schmidt reaction) has not previously been achieved. Such an alginate carrier is beneficial because, as shown by the examples, it allows ovalbumin to be selectively extracted from egg white material.

The azide moiety may, in one embodiment, be an alkali metal azide. In general, azide moieties include sodium azide, potassium azide, iodine azide and bromine azide. A particular preferred azide moiety is sodium azide.

In one embodiment of the method of the seventh aspect, covalently crosslinked calcium alginate with amine functional groups is prepared by mixing covalently crosslinked calcium alginate with carboxylate functional groups (e.g. prepared as above) with a solution of azide salt, e.g. sodium azide. The azide salt, e.g. sodium azide, may suitably be provided in an amount of 0.1 to 3 g per 1 g of calcium alginate, e.g. from 0.2 to 2 g, and especially from 0.3 to 1.5 g, per 1 g of calcium alginate. The azide salt, e.g. sodium azide, may suitably be provided in acidic solution, e.g. HCl.

The reaction may be left to occur at about room temperature and for a period of time from 30 minutes to 12 hours, e.g. 1 to 8 hours or 1 to 4 hours. In one preferred embodiment, the Schmidt reaction is left to occur at about room temperature and for a period of time of 12 hours or longer or 18 hours or longer, such as 24 hours or longer; for example a period of time from 18 hours to 72 hours, or from 24 hours to 48 hours. Periods of time that are 12 hours or longer and especially about 24 hours or longer have been found to result in a crosslinked alginate based carrier that can extract increased amounts of ovalbumin from egg white material.

In one embodiment the covalently crosslinked calcium alginate with carboxylate functional groups used in this method of the seventh aspect has been prepared from ionically crosslinked calcium alginate. In one embodiment, this ionically crosslinked calcium alginate is in the form of beads that have been made using alginate concentrations of 2-5% w/v (e.g. 3-5) and CaCl₂ concentrations of 6-10% w/v (e.g. 8-10). In one such embodiment, the ionically crosslinked calcium alginate is in the form of beads that have been made using an alginate concentration of 5% w/v and CaCl₂ concentration of 10% w/v. Using beads with these amounts of alginate and calcium is particularly useful for selectively extracting ovalbumin.

The invention will now be further described, in a non-limiting fashion, with reference to the following examples.

### EXAMPLES

Materials

Alginic acid sodium salt (M/G=1.56), CaCl₂, NaOH, absolute ethanol 99.8%, epichlorohydrin (99%) and sodium azide were purchased from Merck.

Chicken eggs were purchased from a local supermarket.

All water used was ultrapure, milliQ® deionized water.

Methods

A) Preparation of Ionically Crosslinked Calcium Alginate (Ca-Alg) Beads

Sodium alginate (2-6% w/v in deionized water) was added dropwise to CaCl₂ (2-10% w/v in deionized water) using a syringe with a needle while stirring the CaCl₂ solution to form beads. The resulting beads were left in the CaCl₂ solution in a shaking incubator for 24 h at room temperature to allow the beads to harden. Subsequently, the beads were filtered under vacuum, washed with excess water and stored at 4° C. to provide isolated Ca-Alg beads.

Ca-Alg beads were prepared using different concentrations of Ca and alginate. In this regard, beads were formed using CaCl₂ concentrations of 2, 4, 6, 8, or 10% w/v, and using alginate concentrations of 2, 3, 4, 5 or 6% w/v.

B) Preparation of Covalently Cross-Linked Alginate Beads (CCLABs)

Covalently cross-linked alginate beads (CCLABs) were prepared by replacing the Ca²⁺ in the Ca-Alg beads with the bifunctional linker epichlorohydrin.

In this regard, the Ca-Alg beads (6% w/v alginate with 2% w/v calcium chloride) were added to containers of NaOH (1M) and epichlorohydrin (at concentrations ranging from 1M to 3M) in ethanol (60% v/v in water). The beads were left stirring for 3-24 h at room temperature. The resulting covalently cross-linked beads were filtered under vacuum, washed with ethanol (60% v/v in water), washed with water, and stored at 4° C.

C) Preparation of Amine Functionalized CCLABs (NH₂-CCLABs) Through Schmidt Reaction

A solution of sodium azide (0.3-1.5 g) in HCl (3M in water) was prepared. To this solution was added CCLABs (1 g) as prepared in section B) and the resulting mixture was stirred for 2-24 h at room temperature. This led to amine functionalisation of the covalently cross-linked alginate beads, i.e. the formation of NH₂-CCLABs. These resulting NH₂-CCLABs were filtered, washed with excess milliQ water and stored at 4° C.

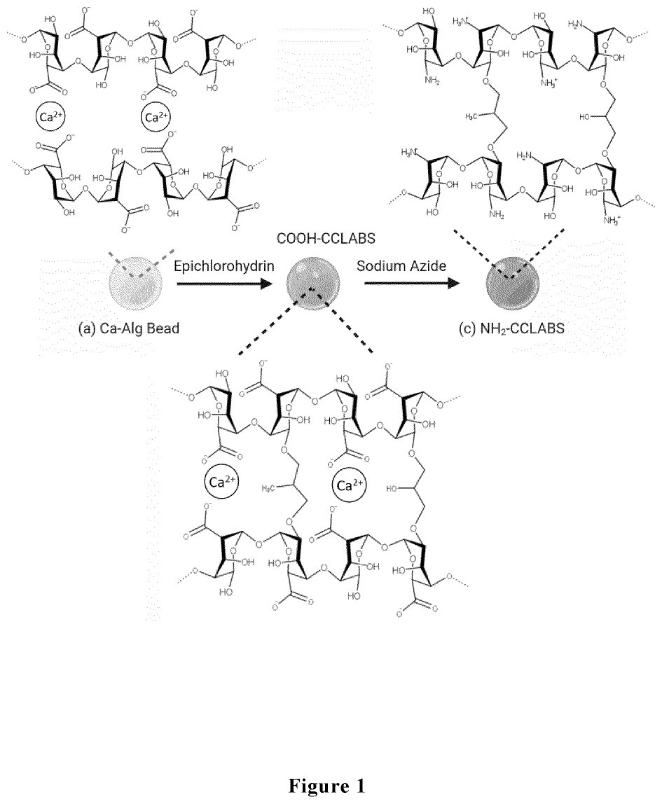

FIG. 1 shows a schematic representation of an exemplary synthesis where Ca-Alg beads are converted to CCLABs and then further converted to NH₂-CCLABs.

D) Analysis of Ca-Alg Beads and CCLABs

The Ca-Alg beads and the CCLABs were characterized by Fourier Transform Infrared (FTIR) and spectra were collected by Perkin Elmer Precisely Spectrum 100 FTIR Spectrometer at a 4000 cm⁻¹ to 650 cm⁻¹ range with 8 scans and a 4 cm⁻¹ resolution.

The surface morphologies of the Ca-Alg beads and the CCLABs were characterized by cryo scanning electron microscopy (cryo-SEM) (Hitachi SU-6600 FESEM with Gatan Cryotransfer stage) at 5 kV accelerating voltage.

Calcium content was determined using an Inductively Coupled Plasma Atomic Emission Spectrometer (ICP-AES; Varian Liberty 150 Qin-AA-1003, USA). Ca-Alg beads (200 mg) were digested using Mars Microwave Digestion containing 65% nitric acid (5 mL). The following parameters were set for Mars One Touch Technology: Control style: Ramp to Temperature, Power: 900 W, Ramp Time: 15.00, Hold Time: 15:00, Temperature: 200° C., Temperature Guard: 260° C., Pressure: 800 Pa.

Bead mass was measured using a high precision balance (Kern, ABS-N/ABJ-NM, Germany).

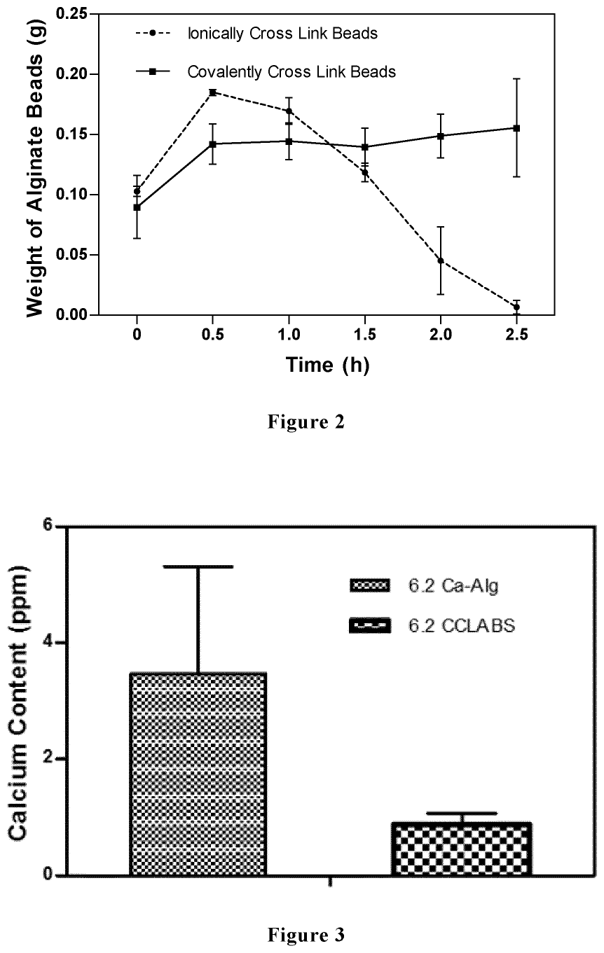

FIG. 2 shows the weight of the Ca-Alg beads (ionically crosslinked) and the CCLABs (covalently crosslinked) over time in an aqueous medium. This illustrates the amount of swelling for the beads over time, and thus the stability of the beads. The Ca-Alg beads are adequately stable in aqueous environment. However, the CCLABs, which are cross-linked with epichlorohydrin, exhibit better structural stability, and take up less water, than the Ca-Alg beads.

FIG. 3 shows a comparison of the calcium content (ppm) of the Ca-Alg beads and of the CCLABs. The CCLABs had significantly less calcium than the Ca-Alg beads: the CCLABs contained 0.88 ppm calcium and the Ca-Alg (2 w/v % Ca, 6 w/v % Alg) beads had 3.46 ppm calcium.

Ca-Alg (2 w/v % Ca, 2 w/v % Alg) beads (not shown) had 0.95 ppm calcium.

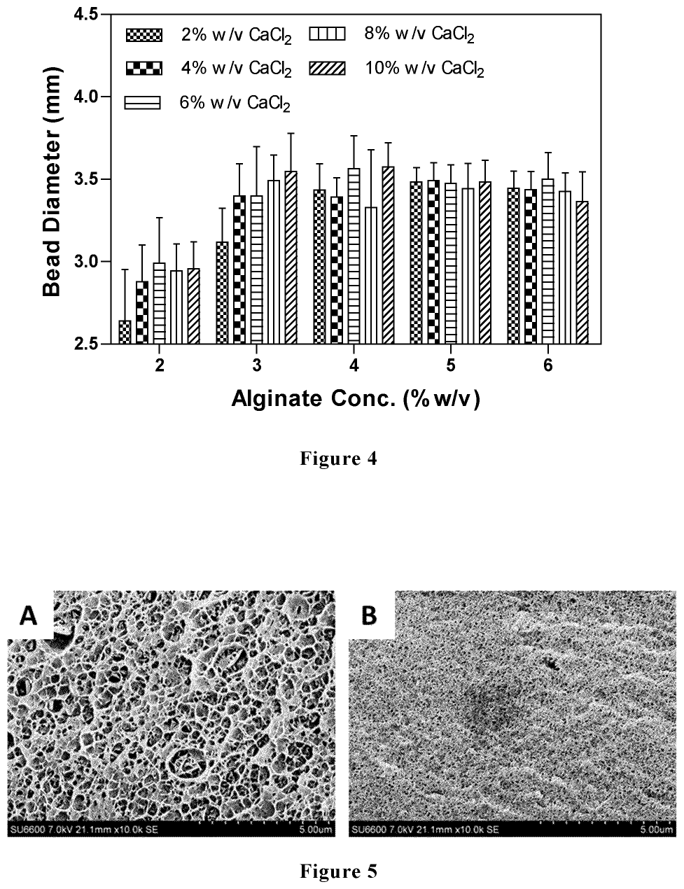

FIG. 4 shows the average bead diameter (mm) of the Ca-Alg beads that have been made using alginate concentrations of 2-6% w/v and CaCl₂ concentrations of 2-10% w/v. The diameters of the Ca-Alg beads were measured visually, using a Keyence Digital Microscope (VHX-500F) and the average (mean number average) diameter obtained. The beads made using 2% w/v alginate had a slightly lower diameter of between 2.5 and 3.0 mm, but the other beads had a diameter of about 3.5 mm.

FIG. 5 shows scanning electron micrographs of cryo-prepared beads: (A) the Ca-Alg beads prepared from 6% w/v alginate and 2% w/v CaCl₂; and (B) the corresponding CCLABs. It can be seen that the CCLABs have a finer pore structure than the Ca-Alg beads.

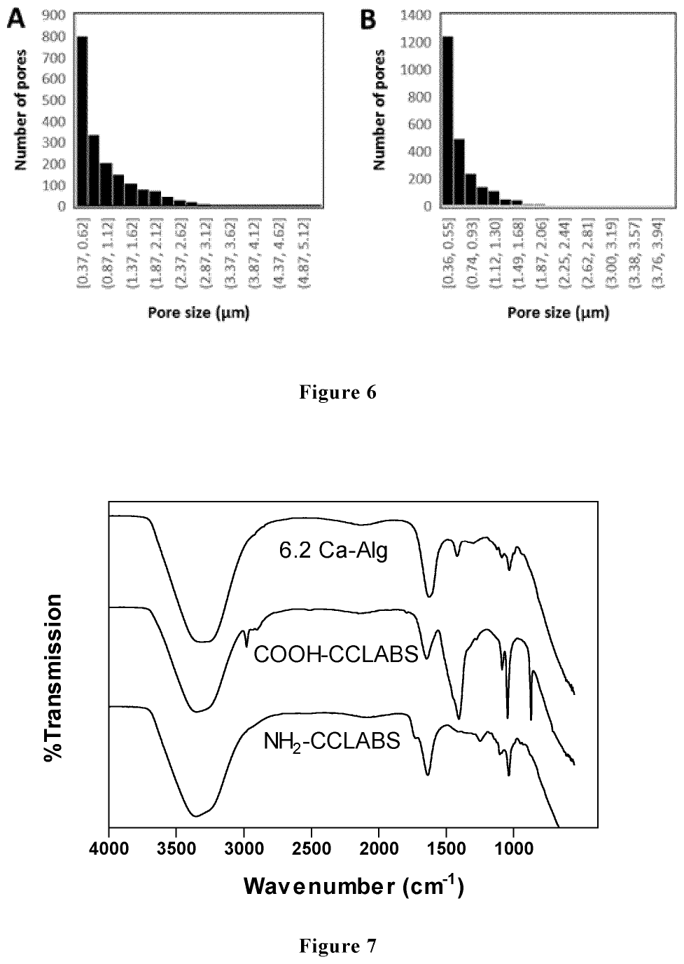

FIG. 6 shows the pore size distributions of the beads as analyzed in FIG. 5. Pore size distributions were determined from the SEM images shown using ImageJ tool. (A) Ca-Alg showed high abundance of pores (˜800) in the range of 0.37 to 0.62 μm in diameter. (B) CCLABS exhibited a higher number of pores (>1200) in the range of 0.36 to 0.55 μm in diameter.

E) Analysis of Ca-Alg Beads, CCLABs and Amine-Functionalised (NH₂—) CCLABs FTIR Analysis was Carried Out on the Ca-alginate Beads, the CCLABs and the NH₂-CCLABs.

FIG. 7 shows the FTIR spectra for Ca-Alg beads, CCLABs and NH₂-CCLABs.
All three spectra show a broad hydroxyl/amine peak (—OH/—NH) at about 3309 cm⁻¹ (Ca-Alg) and 3352 cm⁻¹ (CCLABs and NH₂-CCLABs).
Characteristic absorption peaks representing the stretching bond of a carboxyl functional group (—COOH) were observed at 1627 cm⁻¹ (Ca-Alg beads), 1643 cm⁻¹ (CCLABs) and 1635 cm⁻¹ (NH₂-CCLABs).
Absorption peaks for C−H stretching were observed at 1420 cm⁻¹ (Ca-Alg beads) and 1406 cm⁻¹ (CCLABs).
The peak at 1249 cm ⁻¹ (NH₂-CCLABs) is attributed to C—N stretching.
The peaks at 1032 cm⁻¹, 1044 cm⁻¹ and 1035 cm⁻¹ are attributed to C—O stretching from the ether group (—COC—).

F) Preparation of Egg White Material

Chicken eggs were cracked and the egg yolk was physically separated from the egg white. The egg white was diluted with deionized water, in a ratio of 1 volume of egg white to 6 volumes of water. The pH of the diluted egg white was 9.31.

The diluted egg white was shaken using an orbital shaker for 24 hours at 4° C. and then centrifuged at 4500 rpm for 10 minutes at 4° C. The supernatant contained the diluted egg white protein. A few ml (a drop) of 0.17% (v/v) Tween® 20 (a non-ionic polyethylene glycol sorbitan monolaurate surfactant) was added to the supernatant to maintain the protein solubility.

The pH of the resulting samples was modified by adding citric acid (1M in water) to obtain samples of egg white material having pH values of 5, 7 and 9 respectively. The pH was measured using a pH meter.

G) Extraction of Protein from Egg White Material—Method 1

100 mg portions of beads were added, and immersed into, separate 2 mL samples of the prepared egg white material. One sample had the Ca-Alg beads added, one sample had the CCLABs added and one sample had the NH₂-CCLABS added.

The resulting mixtures were each shaken at 17° C. for 24 h, to allow protein to bind to the beads. The protein loaded beads were removed from the remaining egg white material by filtration. The protein loaded beads were then washed with excess water.

H) Extraction of Protein from Egg White—Method 2

A mixture was prepared of 1 mL of the prepared egg white material plus 1 mL NaCl (50-350 mM in water). A 100 mg portion of Ca-Alg beads was suspended in this 2 mL sample. The resulting mixture was shaken overnight at room temperature to allow protein to bind to the beads.

The protein loaded beads were filtered under vacuum, washed with deionized water and resuspended in 4% w/v NaCl solution for 20 minutes. The protein-loaded beads were then filtered, and the filtrate was retained for SDS-PAGE analysis.

I) Obtaining Unbound Protein

The protein loaded beads from G) and H) were suspended in excess NaCl solution (0.68 M in water) for 5 minutes to release the protein from the beads and obtain a solution of the protein in the salt solution. The saline protein solution, containing unbound protein, was then separated from the beads by filtration.

J) Analysis of Protein Samples Obtained Using Ca-Alg beads, Method 1

The fractions collected in I) by releasing egg white protein from the Ca-Alg beads were analysed by sodium dodecyl polyacrylamide gel (SDS-PAGE). Electrophoresis was run on a 12.5% resolving gel with 5% stacking gel and resolved at 130 V for 70 min followed by 0.1% w/v Coomassie Blue R 250 in acetic acid/methanol/water solution (1:3:6, v/v/v), and de-stained in acetic acid/methanol/water solution (1:3:6, v/v/v).

SDS PAGE gels were examined using Syngene G:Box Chemi XRQ. Lysozome (Lyz) is about 14 kDa, ovomucoid (Ovm) is about 28 kDa, ovalbumin (Oval) is about 48 kDa and ovotransferrin (OTf) is about 76 kDa.

The fractions were also analysed by UPLC. The chromatographic system used was an Agilent 1290 Infinity II (Agilent Technologies) equipped with a quaternary pump, a diode array detector, and an auto-sampler with an injection loop. Optimization of the method was carried out using standard protein solutions (70% OTf solution contains OTf, Oval and Lyz) and analysed in triplicate. Known concentrations of the standard solutions were prepared in 6 M guanidine hydrochloride and further diluted with water containing 0.05% TFA. Separations were performed on reverse-phase analytical column C-18 (BIOshell A400 C18, Merck) with Poroshell packing (3.4 μm particles with 400 Å pores, 2.1 mm×15 cm). A gradient of two mobile phases (A and B) was used. Solution A: 0.05% TFA in water; solution B: 0.05% TFA in acetonitrile. 0 minutes: 15% B to 7 mins: 75% B. Data was collected for 7 mins. Flow rate: 0.3 mL/min, column temperature: 30° C., detection wavelength: 215 nm, injection volume: 2 μL.

The effect of the composition of the beads and the pH of the egg white material was studied.

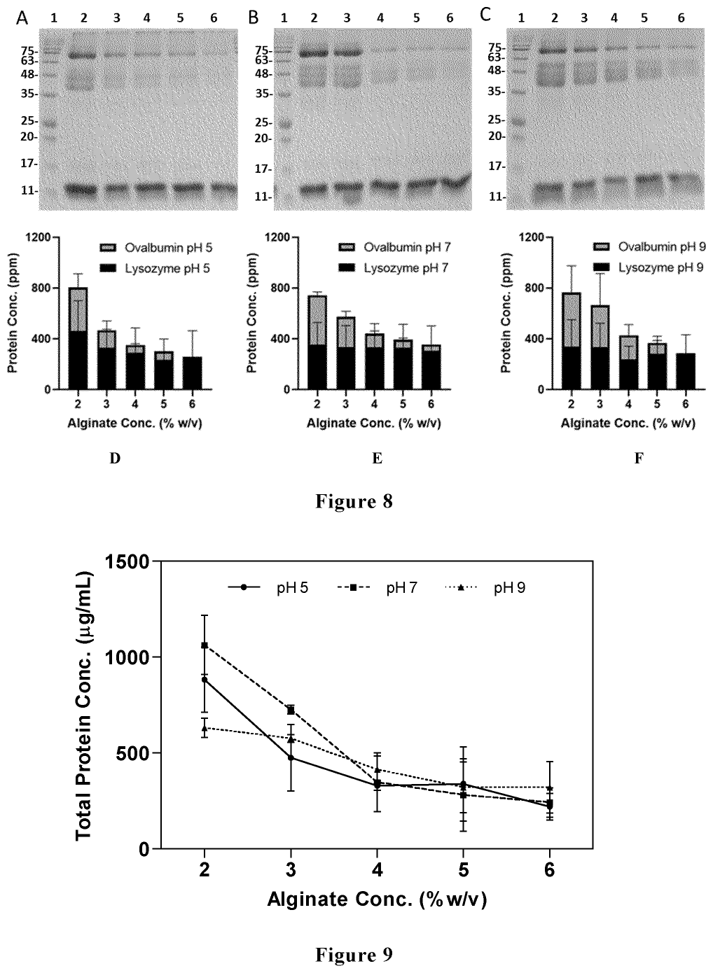

FIG. 8 illustrates the results for proteins extracted from prepared egg white, for five different sets of the Ca-Alg beads and three different incubation conditions in terms of the pH of the prepared egg white. A, B and C are Coomassie Blue stained SDS-PAGE gels. Lane 1 is the protein ladder. Lanes 2-6 correspond to the beads with different alginate concentrations (2, 3, 4, 5 and 6% w/v). D, E and F are corresponding bar charts showing the concentration of Oval and Lyz extracted under the conditions for the SDS-PAGE analysis. The results in A and D are for beads incubated in prepared egg white at pH 5; the results in B and E are for beads incubated in prepared egg white at pH 7, and the results in C and F are for beads incubated in prepared egg white at pH 9.

As the concentration of alginate increases from 2% w/v to 6%, the amount of Oval extracted decreases but the amount of Lyz extracted remains substantially constant. Therefore, as the concentration of alginate increases, the purity of the extracted Lyz increases.

As the pH is increased, the concentration of each protein extracted remains generally constant.

It can therefore be seen there is good selectivity for Lyz by using Ca-Alg beads made with 6% w/v alginate over a range of pH values for the egg white material.

Beads based on 6% w/v alginate with 2% w/v CaCl₂ enabled the largest fraction of Lyz (258.79 μg/ml) to be separated from all egg white proteins.

FIG. 9 shows the total protein concentration extracted against the alginate concentration used to produce each of the Ca-Alg bead compositions, for three different incubation conditions in terms of pH.

When using beads with lower alginate concentrations, more protein was extracted. As the alginate concentration was increased from 2 to 6% w/v, the total protein concentration extracted decreased. This applied for each of the pH values.

For beads with lower alginate concentrations (especially alginate concentrations of 4w/v % or below, such as 2 or 3 w/v %) there was a greater effect associated with changing the pH, whereas at higher alginate concentrations the results were similar at all pH values tested.

The highest total protein concentration extracted was observed when the egg white was at pH 7, followed by pH 5 and then pH 9.

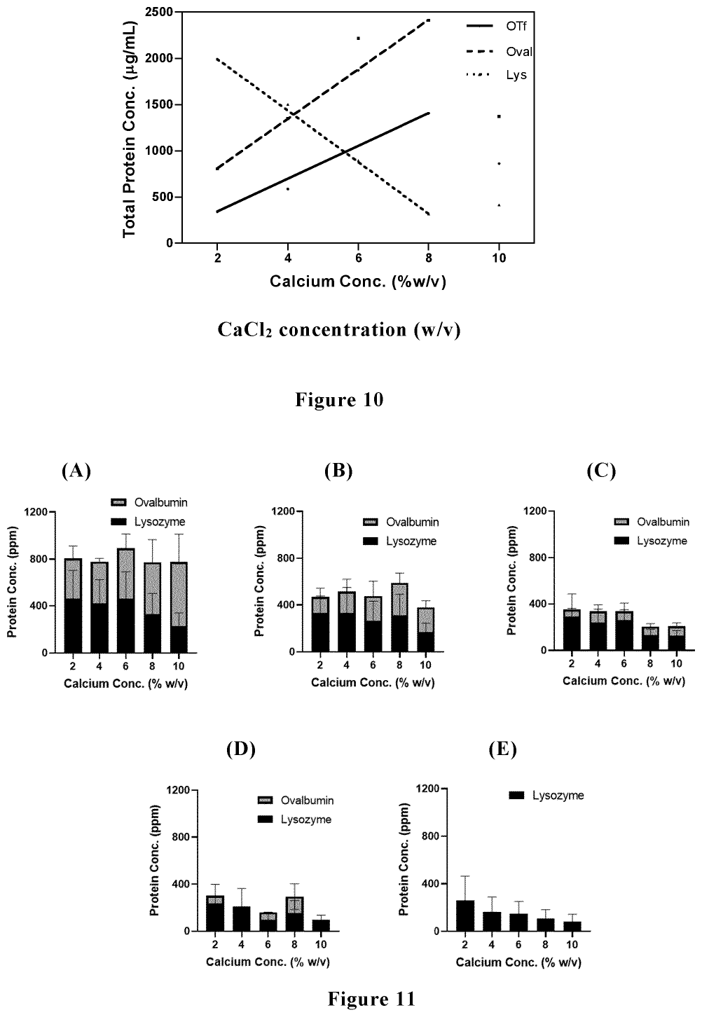

FIG. 10 shows the effect of the CaCl₂ concentration used to produce the Ca-Alg beads on the amount of protein (OTf, Oval and Lyz) extracted by the beads from prepared egg white material that had been adjusted to pH 7, as measured by UPLC and analysed by imageJ.

The results show that increasing the CaCl₂ concentration in the Ca-Alg beads increases the total concentration of OTf and Oval extracted, and decreases the total concentration of Lyz extracted.

Beads formed using a concentration of CaCl₂ of 2-4 w/v % (or lower) selectively extract Lyz.

Beads formed using a concentration of CaCl₂ of 6-8 w/v % (or more) selectively extract Oval and OTf.

FIG. 11 shows the amounts of ovalbumin and lysozyme extracted from prepared egg white, at pH 5, against the calcium concentrations in the Ca-Alg beads used for the extraction, for five different alginate concentrations in the Ca-Alg beads. (A)=2% w/v alginate, (B)=3% w/v alginate, (C)=4% w/v alginate, (D)=5% w/v alginate, and (E)=6% w/v alginate.

As the alginate concentration increases from 2% to 6%, the concentration of extracted protein decreases. However, the effect on ovalbumin extraction is greater than for lysozyme, such that at an alginate concentration of 6% w/v lysozyme was almost exclusively extracted. Thus higher alginate concentrations allow for lysozyme selectivity.

Increasing the calcium concentrations in the beads reduces the concentration of lysozyme extracted.

Therefore the beads with higher alginate and lower calcium are more selective for lysozyme and extract the lysozyme at good concentrations. For example, for beads with an alginate concentration of 6% w/v and 2% w/v CaCl₂, 1.5 mg of Lyz per 1 mL of alginate beads was extracted from the prepared egg white as adjusted to pH 5.

It can therefore be seen there is excellent selectivity and extraction of Lyz by using Ca-Alg beads made with 6% w/v alginate and 2% w/v calcium from egg white material with pH 5.

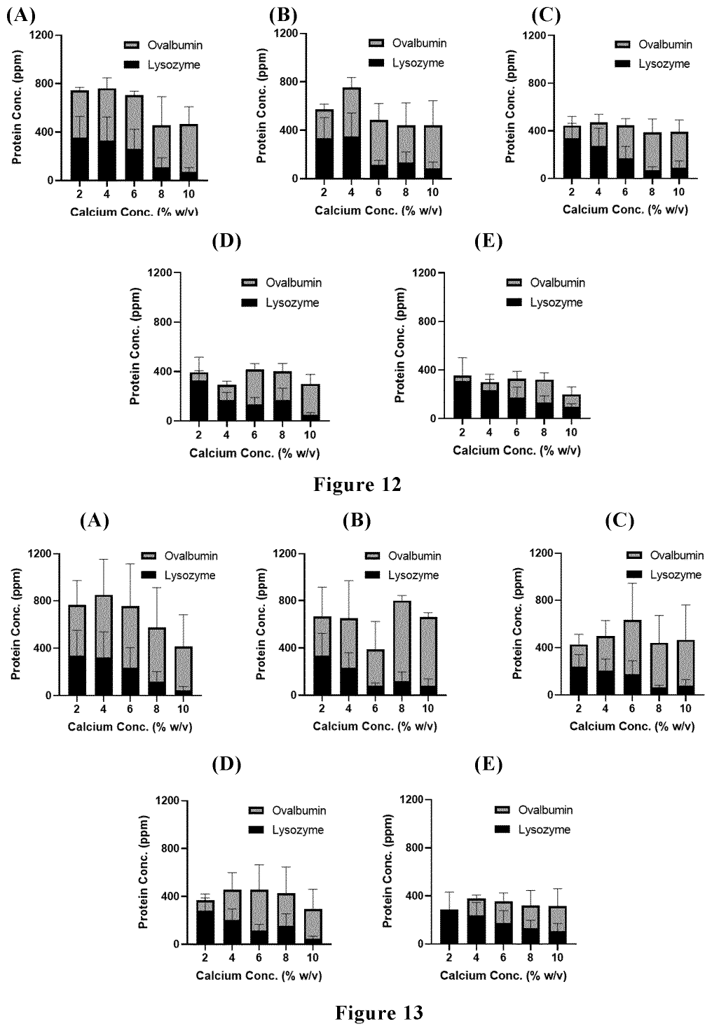

FIG. 12 shows the amounts of ovalbumin and lysozyme extracted from prepared egg white, at pH7, against the calcium concentrations in the Ca-Alg beads used for the extraction, for five different bead types in terms of their alginate concentrations. (A)=2% w/v alginate, (B)=3% w/v alginate, (C)=4% w/v alginate, (D)=5% w/v alginate, and (E)=6%

A similar trend is seen as at pH 5 as shown in FIG. 11: as the alginate concentration increases from 2% to 6%, the concentration of extracted protein decreases but the effect on ovalbumin extraction is greater than for lysozyme, whilst increasing the calcium concentrations in the beads reduces the concentration of lysozyme extracted.

However, the selectivity achieved for lysozyme was not as great at pH 7 as for pH 5.

FIG. 13 shows the amounts of ovalbumin and lysozyme extracted from prepared egg white, at pH 9, against the calcium concentrations in the Ca-Alg beads used for the extraction, for five different bead types in terms of their alginate concentrations. (A)=2% w/v alginate, (B)=3% w/v alginate, (C)=4% w/v alginate, (D)=5% w/v alginate, and (E)=6% w/v alginate.

It can be seen that pH 9 provides better options for selective ovalbumin extraction. Increasing the calcium concentrations in the beads reduces the concentration of lysozyme extracted. At 10% w/v CaCl₂ for beads with 3-5% w/v alginate there was good selectivity for ovalbumin.

Ovalbumin was best separated from the prepared egg white at pH 9 with the beads prepared from 5% w/v alginate with 10% w/v CaCl₂, where the ovalbumin binding capacity was determined to be 1.45 mg/ml.

It can therefore be seen there is excellent selectivity and extraction of Oval by using Ca-Alg beads made with 5% w/v alginate and 10% w/v calcium from egg white material with pH 9.

The effect of the incubation time for the beads in the egg white material was studied.

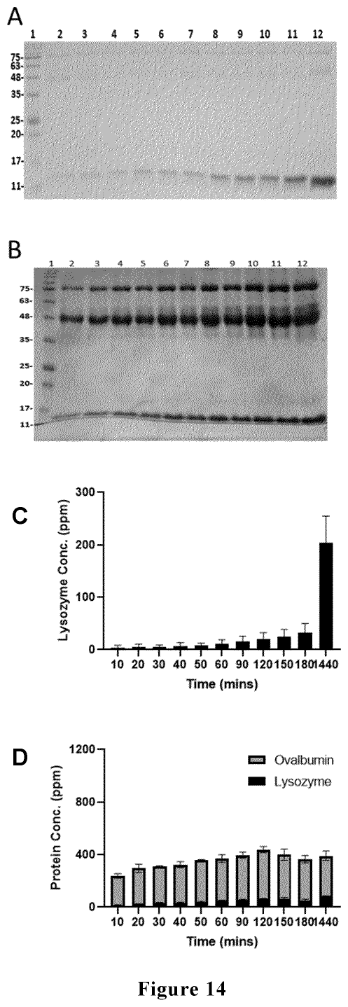

FIG. 14 illustrates the results for protein extracted over time from prepared egg white, for two different sets of Ca-Alg beads and incubation conditions. A and B are Coomassie Blue stained SDS-PAGE gels. Lane 1 is the protein ladder. Lanes 2 to 12 correspond to time points of 10, 20, 30, 40, 50, 60, 90, 120, 150 and 180 minutes, and 24 hours respectively. C and D are corresponding bar charts showing the amount of protein extracted with respect to the incubation time course. A and C are the results for beads prepared from 6% w/v alginate and 2% w/v CaCl₂, when incubated in prepared egg white at pH 5. These are beads and conditions that, based on the FIG. 11 results, would be expected to be more selective for Lyz. B and D are the results for beads prepared from 5% w/v alginate and 10% w/v CaCl₂, when incubated in prepared egg white at pH 9. These are beads and conditions that, based on the FIG. 13 results, would be expected to be more selective for Oval.

For the first set of beads/conditions (see FIGS. 14A and C) there was selectivity for Lyz. The amount of protein extracted increased steadily throughout the 24 hour period. In this regard, 259 μg/ml Lyz was extracted after 24 hours, with negligible amounts of other proteins.

Therefore for extracting Lyz, it has been shown that there is a benefit to having a longer incubation time (i.e. the time period over which the egg white material is in contact with the alginate beads) in order to obtain more Lyz and that this is without detriment to the purity.

For the second set of beads/conditions (see FIGS. 14B and D), there was selectivity for Oval. A concentration of 221 μg/ml Oval was extracted after 10 minutes. Following this, although the levels of Oval extracted increased over the 24 hour period, the gain was only relatively small. In addition, over that time period there was also an increase in the amount of Lyz extracted, although this was also modest. Thus the best selectivity for Oval was observed at 10 minutes and there was not much more Oval extracted by using a longer incubation.

Therefore for extracting Oval, it has been shown that there is a benefit to having a relatively short incubation time in order to obtain the best purity for the extracted Oval and this is without too much detriment to the amount extracted.

K) Analysis of Protein Samples Obtained Using Ca-Alg Beads, Method 2

In method 2 above, saline egg white solutions (prepared egg white material plus NaCl) were used to extract protein using Ca-Alg beads. The effects of the NaCl concentration and the pH of the saline egg white solutions on the amount of ovotransferrin isolated were investigated.

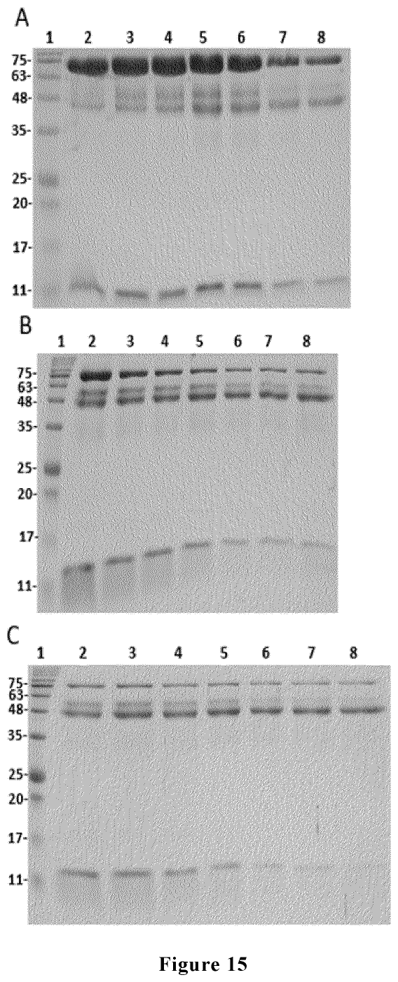

FIG. 15 shows 12.5% SDS PAGE analysis of proteins extraction from egg white mixture containing different NaCl concentrations. Lane 1 is protein ladder, for lane 2 the egg white contains 50 mM NaCl, lane 3 is 100 mM, lane 4 is 150 mM, lane 5 is 200 mM, lane 6 is 250 mM, lane 7 is 300 mM and lane 8 is 350 mM NaCl. (A) shows the results where the egg white material had a pH of 5, for (B) the egg white material had a pH of 7, for (C) the egg white material had a pH of 9; and

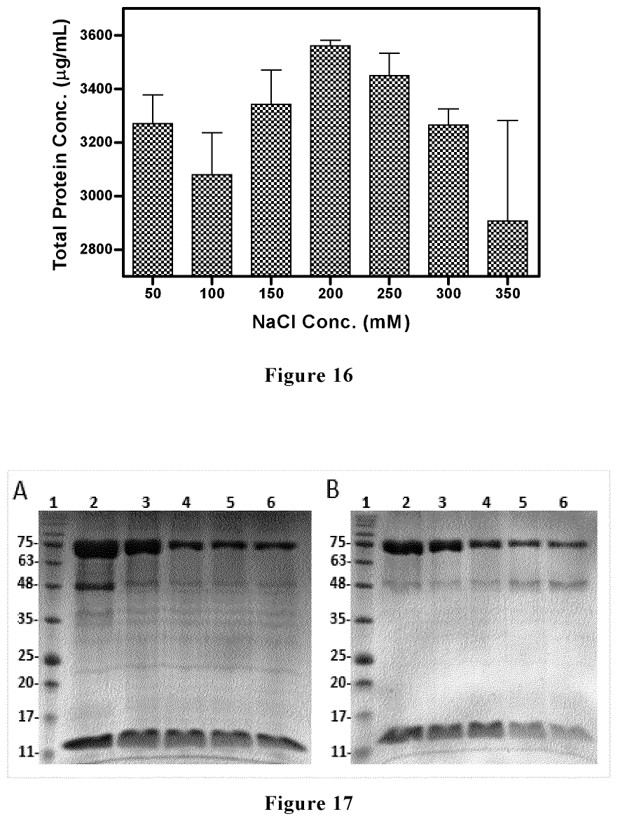

FIG. 16 shows total protein concentration extracted from the pH 5 egg white over a range of Na Cl concentrations.

Unexpectedly, at a NaCl concentration of 50 mM the Ca-Alg beads captured OTf at a level of 3270 g/ml of total protein. NaCl concentrations greater than 200 mM in egg white material at pH 5 resulted in a decrease in the total amount of OTf captured, although these methods were still effective at capturing OTf.

An egg white solution of pH 5 mixed with 50 mM NaCl using Ca-Alg beads made from 3% w/v alginate and 2% w/v CaCl₂ was found to be very effective for extracting OTf.

FIG. 17 shows 12.5% SDS PAGE analysis (Coomassie blue staining) of proteins extracted from an egg white mixture containing 50 mM NaCl concentration. (A) shows results for egg white at pH 5 with 50 mM NaCl, where lane 1 is protein ladder, lane 2 is 2.2 Ca-Alg beads (i.e. Ca-Alg beads made using 2% w/v alginate and 2% w/v CaCl₂), lane 3 is 3.2 Ca-Alg beads (i.e. Ca-Alg beads made using 3% w/v alginate and 2% w/v CaCl₂), lane 4 is 4.2 Ca-Alg beads, lane 5 is 5.2 Ca-Alg beads, lane 6 is 6.2 Ca-Alg beads. (B) shows results for egg white at pH 5 with 50 mM NaCl, where lane 1 is protein ladder, lane 2 is 3.2 Ca-Alg beads, lane 3 is 3.4 Ca-Alg beads, lane 4 is 3.6 Ca-Alg beads, lane 5 is 3.8 Ca-Alg beads, lane 6 is 3.10 Ca-Alg beads.

Using 2.2 Ca-Alg beads, 3285.2 gem' total protein was captured from egg white solution of pH 5 with 50 mM NaCl. Using 3.2 Ca-Alg beads, the results were similar to the results of 2.2 Ca-Alg beads; however, the 3.2 Ca-Alg beads captured 2492.8 gem' total protein with higher purity OTf than 2.2 Ca-Alg beads as no Oval was captured. Further increasing the concentration of alginate (4-6% w/v), there is a decrease of OTf captured (699.2-1416.5 μg/ml).

3% w/v alginate beads were found to be mechanically stronger beads than 2.2 Ca-Alg beads.

In general, Ca-Alg beads made with a lower calcium chloride concentration (such as 2 to 4% w/v) are preferable as these extract higher total protein concentrations.

In general, increasing the relative amount of egg white (EW) to NaCl (by volume) slightly decreases the total protein concentration extracted from the egg white material.

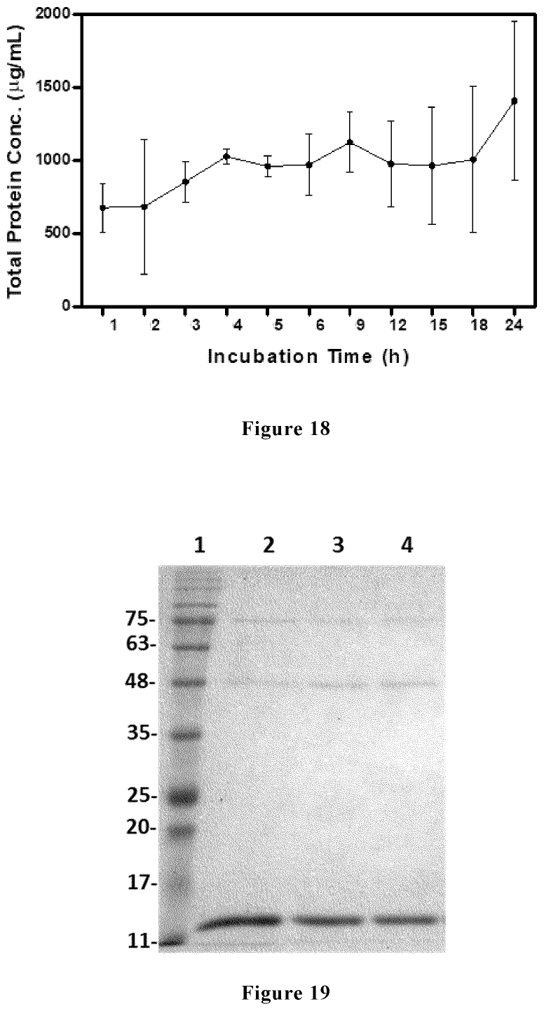

FIG. 18 shows the total protein concentration obtained following incubation over a range of time periods up to 24 hours.

It can be seen that the incubation time (i.e. the time period over which the egg white material is in contact with the alginate beads) has an effect on the quantity of protein isolated. Incubation of egg white material at pH 5, containing 50 mM NaCl and using 3.3 Ca-Alg beads for 24 h captured the highest quantity of OTf.

In summary, it has been shown that by adding salt (NaCl) to an aqueous egg white material, the commercially valuable protein OTf can be isolated in a simple manner from the egg white.

Approximately 90% of OTf present in the natural egg white (2,760 μg/ml) was able to be captured, showing a binding capacity of 4.0 mg/ml.

L) Analysis of Protein Samples Obtained Using CCLABs

The fractions collected by extracting egg white protein from CCLABs were analysed, in a similar manner to the analysis of fractions obtained using Ca-Alg beads as discussed above. The tested CCLABs were 6% w/v alginate with 2% w/v CaCl₂ beads reacted with 1M epichlorohydrin for 24 hrs unless indicated otherwise.

The effect of the composition of the beads and the pH of the egg white material was studied.

FIG. 19 shows Coomassie Blue stained SDS-PAGE gels of proteins extracted from prepared egg white material, adjusted to different pH values, using the CCLABs. Lane 1 is the protein ladder. Lane 2 shows protein extracted at pH 5; lane 3 shows protein extracted at pH 7; and lane 4 shows protein extracted at pH 9; and

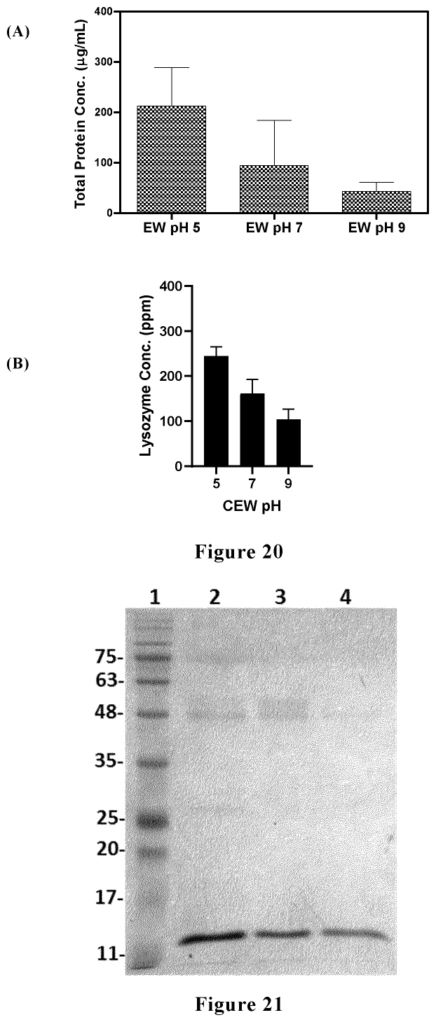

FIG. 20 shows (A) the total protein and (B) the concentration of Lyz extracted from egg white material, adjusted to pH 5, 7 and 9, by using the CCLABs, as measured using the Bradford assay.

It can be seen that the most protein is extracted at pH 5, then pH 7, then pH 9.

The results show that by incubating the CCLABs in egg white material at different pH values, Lyz can be captured in significant amounts, with only low amounts of other proteins present.

The highest amount of Lyz extracted was from prepared egg white at pH 5 (245 μg/ml) with the CCLABs exhibiting a binding capacity of 1.37 mg/ml. Increasing the pH to 9 led to a decrease in Lyz extracted (104 μg/ml).

Significantly, therefore, by using the CCLABs the protein Lyz is obtained selectively, and in relatively pure form, from egg white.

The effect of the composition of the CCLAB beads was studied.

Firstly, tests were carried out where the concentration of epichlorohydrin used for the covalent crosslinking of the beads was varied (1, 2 and 3M).

FIG. 21 shows Coomassie Blue stained SDS-PAGE gels of proteins extracted from prepared egg white using CCLABs that had been prepared using different concentrations of epichlorohydrin. Lane 1 is the protein ladder. Lane 2 shows protein extracted using CCLABs prepared with a 1M solution of epichlorohydrin; lane 3 shows protein extracted using CCLABs prepared with a 2M solution of epichlorohydrin; and lane 4 shows protein extracted using CCLABs prepared with a 3M solution of epichlorohydrin.

It can be seen that over the tested epichlorohydrin concentration range, of from 1M to 3M, the CCLABs were all able to capture Lyz, with low amounts of other proteins present. Therefore the selectivity for Lyz is retained.

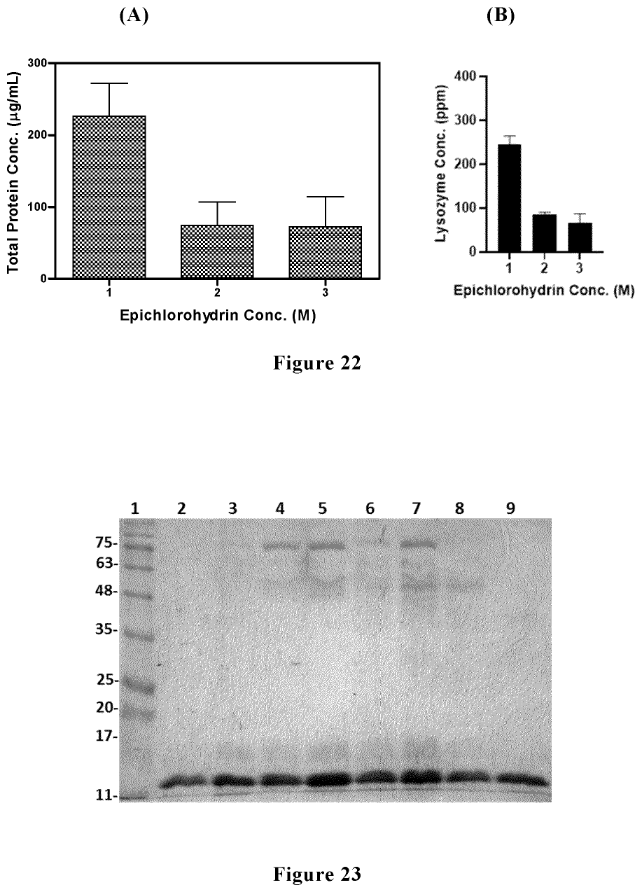

FIG. 22 shows (A) the total protein concentration and (B) the Lyz concentration extracted using the CCLABs prepared with a 1M, 2M or 3M solution of epichlorohydrin, as determined using the Bradford assay.

It can be seen that the most total protein was extracted using CCLABs prepared with a 1M solution of epichlorohydrin, and that the most Lyz was also extracted using such CCLABs.

Preparation with 1M epichlorohydrin extracted 245 μg/ml Lyz, as compared to 67 μg/ml for the CCLABs prepared with 3M epichlorohydrin.

Secondly, tests were carried out in which the length of time over which the crosslinking with epichlorohydrin was carried out was varied (3, 6, 9 and 24 hours).

In each case the CCLABs extracted Lyz in significant amounts (˜675 μg/ml of total protein) from the egg white material and only low amounts of other proteins. The amount of protein extracted increased slightly when using beads that had been crosslinked for 24 hours as compared to the shorter time periods.

Therefore a range of different CCLABs were all able to capture Lyz in large amounts, with low amounts of other proteins present.

Further tests were carried out to assess the ability to reuse the CCLABs, with the CCLABs being used to perform multiple sequential extractions. The unloaded beads were washed with water to remove residual NaCl and subsequently re-immersed in the aqueous egg white material for another separation cycle.

FIG. 23 shows Coomassie Blue stained SDS-PAGE gels of proteins extracted from prepared egg white material using CCLABs after each of eight cycles. Lane 1 is the protein ladder. Lanes 2 to 9 shows the protein extracted from each of cycles 1 to 8 respectively; and

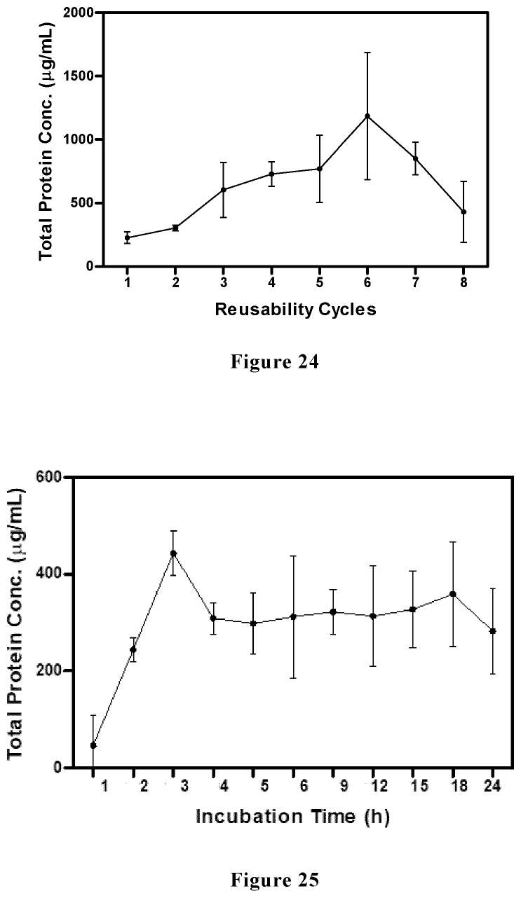

FIG. 24 shows the total protein concentration obtained following each cycle.

Lyz was a large proportion of the proteins extracted. It can be seen that the total protein concentration as extracted increased from cycle 1 to cycle 6; it then decreased from cycle 6 to cycle 8 but still remained at a useful level. Therefore, CCLABs can be re-used for multiple extraction cycles.

Tests were also carried out to assess the effect of incubation time, i.e. the time period over which the egg white material is in contact with the covalently crosslinked alginate beads.

FIG. 25 shows the total protein concentration obtained following incubation over a range of time periods; and

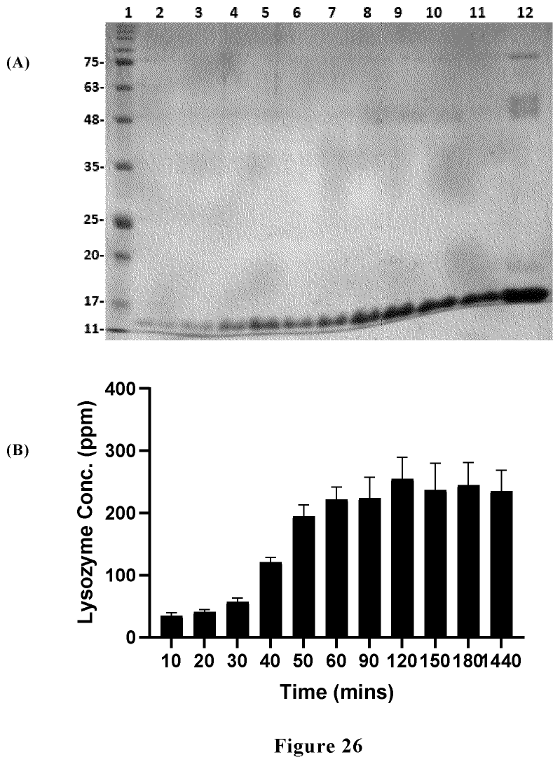

FIG. 26 shows (A) Coomassie Blue stained SDS-PAGE gels and (B) the Lyz concentration obtained following incubation with CCLABs over a range of time periods.

Incubation time has an effect on the total quantity of protein isolated. The total protein concentration that was extracted increased as the incubation period extended from 1 h to 3 h and peaked at 3 h. It then stayed fairly constant from 4 h to 24 h.

Incubation time also has an effect on the quantity of Lyz isolated. The Lyz concentration increased significantly as the incubation period extended from 10 minutes to 1 h, and continued to increase slightly to 2h, but thereafter stayed fairly constant from 2h to 24h.

Therefore there is a benefit to using a long enough incubation time, but about 1-2 hours is both sufficient and optimal to extract Lyz.

The CCLABs captured Lyz at a faster rate than the Ca-Alg beads (see results of FIG. 14).

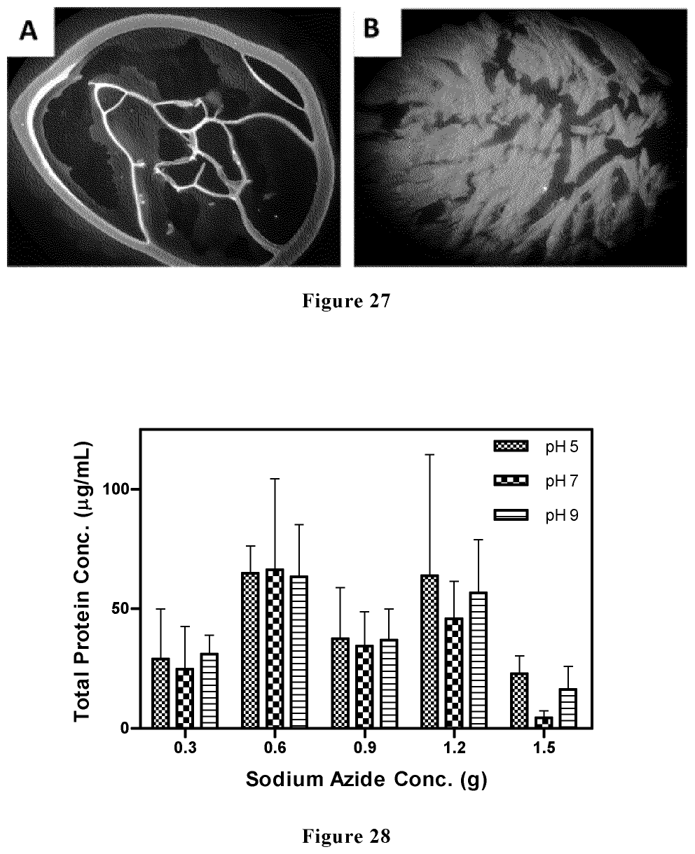

FIG. 27 shows micrographs obtained from using fluorescence microscopy to view Ca-Alg beads and CCLABs which have been contacted with fluorescently tagged lysozyme.

This shows that the lysozyme binds to both Ca-Alg beads and CCLABs, but binds at higher levels to the CCLABs. The lower calcium content of the CCLABs, relative to the Ca-Alg beads, and/or the difference in pore sizes, could explain why Lyz was more selectively captured by the CCLABs beads.

M) Analysis of Protein Samples Obtained Using NH₂-CCLABs

The fractions collected by extracting egg white protein from the NH₂-CCLABs were analysed, in a similar manner to the analysis of fractions obtained using Ca-Alg beads as discussed above.

The conversion of the carboxylate group on the alginate to a primary amine, through the Schmidt reaction, allowed the capture of Oval from the egg white material.

The effect of the composition of the beads and the pH of the egg white material was studied.

FIG. 28 shows the total protein concentration extracted from egg white material by using NH₂-CCLABs, as measured using the Bradford assay. The NH₂-CCLABs beads as tested had been modified using a range of different azide concentrations, from 0.3 to 1.5 g, and the tests were carried out using egg white material with its pH adjusted to pH 5, 7 and 9.

Good results were obtained for beads modified with 0.6-1.2 g of NaN₃. The highest total amount of protein was extracted using beads modified with 0.6 g of NaN₃.

Good results were obtained for the NH₂-CCLABs beads at all of the tested pH values, but, in general, the most protein was extracted from egg white at pH 5.

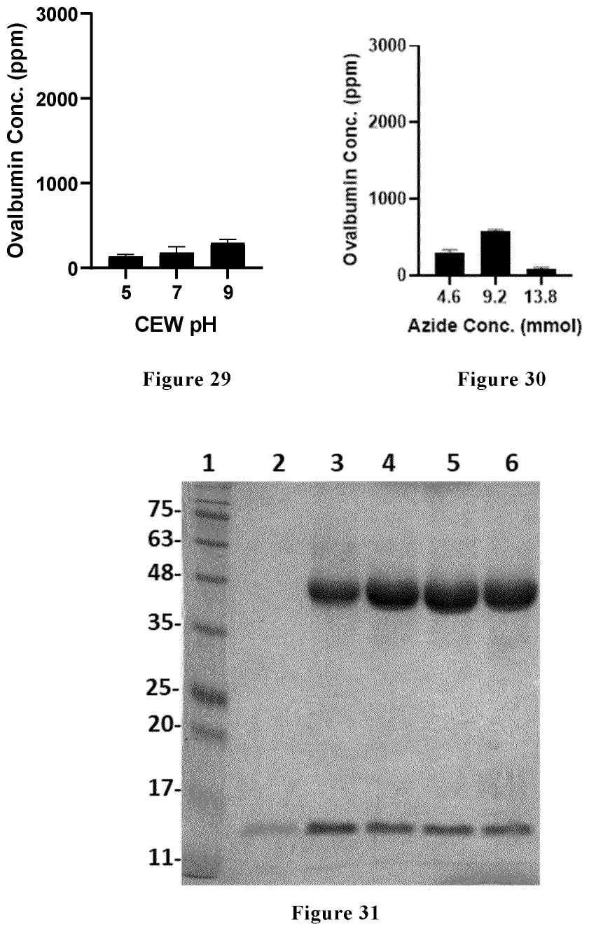

FIG. 29 shows the concentration of ovalbumin extracted from prepared egg white by using NH₂-CCLABs, where the egg white material had its pH adjusted to pH 5, 7 and 9.

It can be seen that Oval is extracted by using the NH₂-CCLABs beads at all tested pH values, but this increases with increasing pH and the most Oval was extracted at pH 9 (298 μg/ml).

FIG. 30 shows the concentration of ovalbumin extracted from prepared egg white using NH₂-CCLABs, where these beads were prepared using differing concentrations of azide.

It can be seen that Oval is extracted by all the tested NH₂-CCLABs beads, but the most Oval was extracted by the beads formed using 9.2 mmol azide, followed by 4.6 mmol azide and then 13.8 mmol azide.

FIG. 31 shows SDS PAGE analysis (Coomassie blue staining) of proteins captured by the amine-modified CCLABs from pH 9 egg white medium. The NH₂-CCLABs beads as tested had been prepared using different reaction times (0, 2, 4, 6 and 24 h) between the NaN₃ and CCLABs. Lane 1 is the protein ladder; lane 2 is beads that had reacted for 0 h (i.e. the beads were still COOH-CCLABS), lane 3 is beads that had reacted for 2 h, lane 4 is beads that had reacted for 4 h, lane 5 is beads that had reacted for 6 h and lane 6 is beads that had reacted for 24 h.

It can be seen that for all of the amine-modified CCLABs (i.e. the beads where the reaction time was greater than zero) a large amount of Oval is extracted from the prepared egg white material. Significantly, there were low amounts of other proteins extracted, i.e. there was selectivity for Oval.

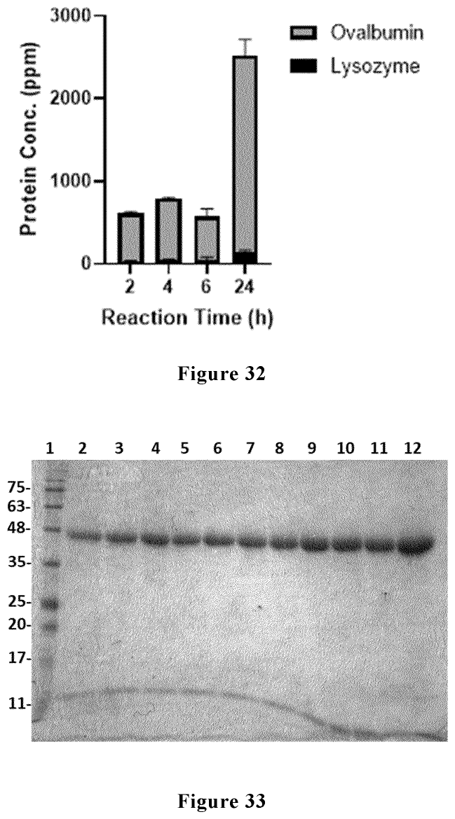

FIG. 32 shows the concentrations of Lyz and Oval captured by the amine-modified CCLABs from prepared egg white at pH 9. The NH₂-CCLABs beads as tested had been prepared using different reaction times (2, 4, 6 and 24 h) between the NaN₃ and CCLABs.

The concentration of Oval recovered was similar for the beads reacted for 2h, 4h and 6h, at about 800 μg/mL. However, there was a clear increase in Oval recovered, up to a peak concentration of about 2500 μg/mL, for beads that had been reacted for 24h. The peak corresponded to a binding capacity of 14.5 mg/ml. This shows that it can be beneficial to ensure that the reaction between the azide and the CCLABs reaches completion in order to maximize the amount of protein that can be extracted.

The total concentration of Lyz recovered was low for all of the tested NH₂-CCLABs beads, i.e. there was selectivity for Oval.

The effect of the incubation time for the beads in the egg white material was studied.

FIG. 33 shows SDS PAGE analysis (Coomassie blue staining) of proteins extracted from prepared egg white at pH 9 using amine-modified CCLABS prepared from 6% w/v alginate, 2% w/v CaCl₂, and cross-linked with 1 M epichlorohydrin, for a number of different incubation times. Lane 1 is the protein ladder. Lane 2 is 1 h, lane 3 is 2 h, lane 4 is 3 h, lane 5 is 4 h, lane 6 is 5 h, lane 7 is 6 h, lane 8 is 9 h, lane 9 is 12 h, lane 10 is 15 h, lane 11 is 18 h and lane 12 is 24 h; and

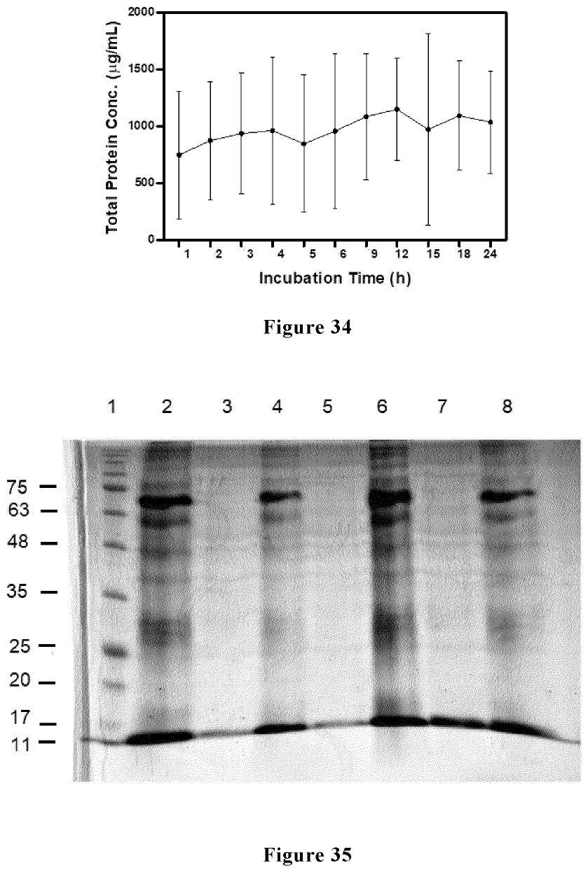

FIG. 34 shows the total protein concentration obtained following the incubation over this range of time periods.

It can be seen that for all of the incubation times a large amount of Oval is extracted from the prepared egg white material when using the NH₂-CCLABs beads. Significantly, there were low amounts of other proteins extracted, i.e. there was selectivity for Oval.

In addition, the incubation time (i.e. the time period over which the egg white material is in contact with the covalently crosslinked amine functionalised alginate beads) has an effect on the quantity of protein isolated. The total protein concentration as extracted increased with incubation time until it reached a peak after 12 hours incubation; then it stayed fairly constant from 18 h to 24h.

Therefore there is a benefit to using a long enough incubation time, but about 12 hours is both sufficient and optimal to extract Oval.

Selective Extraction of Aprotinin from Bovine Lung Solution

Aprotinin is similar to lysozyme in terms of Mw and pI, with a molecular weight of 10.9 kDa and pI of 10.5.

Tests were therefore carried out to see whether aprotinin could be selectively extracted in a similar manner to lysozyme.

Bovine lung solution was used, as a known source of aprotinin.

The beads selected for aprotinin were Ca-Alg beads prepared from 6% w/v alginate and 2% w/v CaCl₂, which had been shown to be effective for selectively extracting lysozyme.

FIG. 35 shows SDS PAGE analysis (Coomassie blue staining) of protein extraction from bovine lung (BL): lane 1 is protein ladder, lane 2 is BL solution, lane 3 is 6.2 Ca-Alginate beads in BL, lane 4 is BL filtrate, lane 5 is 5.10 Ca-Alginate beads in BL, lane 6 is BL filtrate, lane 7 is 6.2 CCLABs in BL, lane 8 is BL filtrate.

The band eluting at ˜11 kDa shows selective extraction of aprotinin.

1. A method of extracting protein from a protein source material, wherein the method comprises:
a) contacting a crosslinked alginate-based carrier with the protein source material and allowing the protein to bind to the carrier, so as to form a protein-loaded carrier product; and
b) separating the protein-loaded carrier product from the remaining protein source material;
wherein the protein source material comprises a mixture of two or more naturally-occurring proteins and water.

2. The method according to claim 1 wherein the protein source material is egg white material; or milk-based material; or animal organ extract material, such as bovine lung solution.

3. The method according to claim 1, wherein the protein source material has undergone one or more treatment selected from: extraction (e.g. extraction of one or more protein), dilution (e.g. dilution with aqueous medium), and addition of one or more agent (e.g. addition of one or more water-soluble salt and/or addition of one or more surfactant and/or addition of one or more acid).

4. The method according to claim 1, wherein the protein is ovalbumin and wherein the crosslinked alginate based carrier is (i) covalently crosslinked alginate having amine functional groups; or (ii) ionically crosslinked calcium alginate formed using an alginate concentration from 1% to 6% (w/v) and a calcium concentration from 6% to 12% (w/v).

5. The method according to claim 4, wherein option (i) applies and the method comprises allowing the protein to bind to the carrier in step a) for a period of time of 10-12 hours or more; or wherein option (ii) applies and the method comprises allowing the protein to bind to the carrier in step a) for a period of time of 10 minutes to 1 hour.

6. The method according to claim 4, wherein the protein source material is egg white material.

7. The method according to claim 6, wherein the pH of the egg white is, or has been altered to be, in the range of 7 or more, such as from 8 to 10, e.g. 9.

8. The method according to claim 1, wherein the protein is lysozyme and wherein the crosslinked alginate based carrier is (i) covalently crosslinked alginate having carboxylate functional groups; or (ii) ionically crosslinked calcium alginate formed using an alginate concentration from 4 to 8% (w/v) and a calcium concentration from 1 to 5% (w/v).

9. The method according to claim 8, wherein option (i) applies and the method comprises allowing the protein to bind to the carrier in step a) for a period of time of 1-2 hours or more; or wherein option (ii) applies and the method comprises allowing the protein to bind to the carrier in step a) for a period of time of 12-24 hours or more.

10. The method according to claim 8, wherein the protein source material is egg white material.

11. The method according to claim 10, wherein the pH of the egg white is, or has been altered to be, in the range of 7 or less, such as from 4 to 6, e.g. 5.

12. The method according to claim 1, wherein the protein source material is egg white that has been diluted with aqueous medium.

13. The method according to claim 12, wherein the aqueous medium contains one or more water-soluble salt, such as sodium chloride, and wherein the crosslinked alginate based carrier is ionically crosslinked calcium alginate having carboxylate functional groups, and wherein the protein is ovotransferrin.

14. The method according to claim 1, wherein the protein is aprotinin and wherein the crosslinked alginate based carrier is (i) covalently crosslinked alginate having carboxylate functional groups; or (ii) ionically crosslinked calcium alginate formed using an alginate concentration from 4 to 8% (w/v) and a calcium concentration from 1 to 5% (w/v).

15. The method according to claim 14, wherein the protein source material is bovine lung solution.

16. The method according to claim 1, wherein the crosslinked alginate based carrier is calcium alginate.

17. The method according to claim 1, wherein the crosslinked alginate based carrier is in the form of beads.

18. A protein-loaded carrier product, comprising protein bound to a crosslinked alginate-based carrier, wherein the protein-loaded carrier product is obtainable by (e.g. has been produced by) the method as defined in claim 1.

19. A method of providing protein in unbound form, the method comprising:
i) providing protein-loaded carrier product as defined in claim 18; and
ii) removing protein from the crosslinked alginate-based carrier so as to provide protein in unbound form.

20. (canceled)

21. (canceled)

22. (canceled)

23. (canceled)

24. (canceled)

25. (canceled)

26. (canceled)

27. (canceled)

28. (canceled)

29. (canceled)

30. The method according to claim 1, wherein the method provides protein in unbound form, and wherein the method further comprises:
c) removing protein from the crosslinked alginate-based carrier so as to provide protein in unbound form.
